(19) United States
(12) Patent Application Publication
(10) Pub. No.: US 2008/0255206 A1
(43) Pub. Date: Oct. 16, 2008

(54) Rxr Agonists and Antagonists, Alone or in Combination with Ppar Ligands, in the Treatment of Metabolic and Cardiovascular Diseases

(76) Inventor: Werner Bollag, Basel (CH)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/091,331

(22) PCT Filed: Oct. 13, 2006
(86) PCT No.: PCT/EP06/09899

§ 371 Date: Apr. 24, 2008

(30) Foreign Application Priority Data
Oct. 25, 2005 (EP) 05023232.1

(51) Int. Cl. A61K 31/4439; C07C 57/03; A61K 31/19; A61K 31/192; A61K 31/381; A61P 3/04; A61P 3/10; C07D 333/24; C07C 57/42; A61K 31/216; A61K 31/443

(52) U.S. Cl. 514/342; 562/500; 514/572; 514/569; 562/492; 549/79; 514/438; 562/496; 514/532; 514/512

(57) ABSTRACT

The present invention relates to the use of one or more retinoid agonists and/or antagonists comprising retinoids with selective Retinoid X Receptor (RXR) agonistic or antagonistic activity alone or in combination with one or more peroxisome proliferator activated receptor (PPAR) ligands for the manufacture of a medicament for the (preferably oral or topical) treatment (this term including prevention/prophylaxis and/or therapy) of one or more manifestations of metabolic syndrome (also known as syndrome X), also called diseases hereinafter, especially from one or more manifestations thereof selected from the group consisting of diabetes type II, obesity, dyslipidemia, hypertension and polyneuropathy, each of which can also be linked with a high risk of cardiovascular diseases. Corresponding methods, the compounds and combinations for use in the treatment of the mentioned diseases and comparable invention embodiments are also described.

### SUMMARY OF THE INVENTION

The present invention relates to the use of one or more retinoid agonists and/or antagonists comprising retinoids with selective Retinoid X Receptor (RXR) agonistic or antagonistic activity alone or in combination with one or more peroxisome proliferator activated receptor (PPAR) ligands for the manufacture of a medicament or (combination) product for the treatment (including prevention/prophylaxis and/or therapy) of one or more manifestations of metabolic syndrome (also known as syndrome X), also called diseases hereinafter, especially from one or more manifestations thereof selected from the group consisting of diabetes type II, obesity, dyslipidemia, hypertension and polyneuropathy, each of which can also be linked with a high risk of cardiovascular diseases. It relates, as well, to one or more RXR agonists and/or antagonists, alone or in combination with one or more PPAR ligands, in the treatment of one or more of the mentioned diseases, to the use of one or more of the mentionned compounds or combinations in the treatment of one or more of these diseases, to a method of treatment of said diseases comprising administering one or more such compounds or combinations to a warm-blooded animal, especially a human, and/or to a pharmaceutical composition or combination product for use in the treatment of any one or more of said diseases comprising one or more such RXR agonists and/or RXR antagonists alone or in combination with PPAR ligands, as well as to combination products of one or more RXR agonists and/or antagonists with one or more PPAR ligands.

### BACKGROUND OF THE INVENTION

Retinoids are a class of compounds structurally related to vitamin A, comprising natural and synthetic compounds. A series of retinoids have been found to be clinically useful mainly in the treatment of dermatological and oncological diseases.

The activity of retinoids is thought to be mediated by the nuclear retinoid receptors RARα, β, γ and/or RXR α, β, γ belonging to the superfamily of steroid, thyroid hormone, vitamin D and peroxisome proliferator-activated receptors. Retinoids with receptor agonistic activity bind and activate retinoid receptors. Retinoids with receptor antagonistic activity bind receptors but do not activate them.

Retinoids are clinically useful in the treatment of various dermatological diseases, such as acne, psoriasis and other keratinizing dermatoses and in the prevention and therapy of some premalignant and malignant diseases.

The efficacious drugs such as all-trans retinoic acid, 13-cis retinoic acid, etretinate, acitretine and tazarotene, are all belonging to the group of compounds that bind and activate nuclear retinoid receptors RAR α, β, γ and are therefore called RAR agonists or retinoids with RAR agonistic activity.

Experimentally, retinoids with retinoid receptor RAR antagonistic activity (retinoid antagonists) are effective in counteracting many properties of retinoids with retinoid receptor agonistic activity (retinoid agonists) such as inhibition of cell proliferation, induction of cell differrentiation, induction of apoptosis and inhibition of angiogenesis (see e.g. Bollag et al., Int.J.Cancer 70, 470-472 (1997). Retinoid antagonists are also suppressing toxic side effects of retinoid agonists such as the signs and symptoms of the hypervitaminosis A syndrome and teratogenesis (see e.g. Standeven et al., Toxicol. Appl. Pharmacol. 138, 169-175 (1996); Eckhardt and Schmitt. Toxicol. Letters 70, 299-308 (1994).

Retinoid antagonists have, therefore, been proposed for clinical use in prevention and therapy of retinoid-induced toxicity and side effects, particularly of the so-called hypervitaminosis A syndrome.

Furthermore, retinoids with retinoid receptor RXR antagonistic activity have been found to be efficacious in experimental models predictive for the treatment of T-helper cell type 2 (Th2)-mediated immune diseases, or immunoglobulin E (IgE)-mediated diseases. allergic diseases, atopic diseases or diseases mediated by the Th2-related cytokines. They encompass atopic dermatitis (neurodermitis), allergic rhinitis or hay fever and allergic bronchial asthma (see e.g. WO 99/24024 and WO 00/53562).

Retinoids with retinoid receptor RXR antagonistic activity have also been shown to be efficacious in model systems for osteoporosis (see e.g. WO 00/53562). In addition, RXR antagonists, are useful in the treatment of multiple sclerosis and in the treatment of inflammatory diseases of the skin and/or mucous membranes, and especially of other tissues and organs, especially of inflammatory diseases of bones and/or joints, by all kinds of pharmaceutical administration, but in particular by oral or by topical application e.g. to the skin and mucous membranes or further parenterally as described in co-pending patent applications. “RXR antagonist treatment against multiple sclerosis” and “RXR antagonists in the treatment of inflammatory diseases” (see PCT/EP2005/007762 and PCT/EP2005/007763).

### GENERAL DESCRIPTION OF THE INVENTION

For the first time, quite unexpectedly, it has now been found that certain RXR agonists and RXR antagonists administered as one or more single agents or especially in combination with one or more PPAR ligands are useful in the prevention and treatment of metabolic and cardiovascular diseases falling under what is named the metabolic syndrome (or syndrome X) such as diabetes type II, obesity, dyslipidemia, hypertension and/or atherosclerosis by all kinds of pharmaceutical administration, preferably by systemic, especially oral administration and in special cases by topical application, e.g. for promotion of wound healing in diabetic patients.

As part of this invention it is shown herein in experimental investigations that a number of RXR agonists, as well as a number of RXR antagonists exert a favourable effect on glucose metabolism, reducing serum glucose levels. In addition, the preferred compounds of these classes decrease triglycerides and increase HDL-cholesterol in blood, thus providing evidence that such compounds, due to their mentioned favourable influence on glucose and lipid metabolism, are especially useful in the treatment of diseases falling under the generic term metabolic syndrome, especially diabetes type II, obesity, dyslipidemia and atherosclerosis.

A series of publications on experimental and clinical investigations have appeared which have shown that various PPAR ligands (α,β/δ and γ) have a favourable effect on glucose metabolism as insulin sensitizing drugs and on lipid metabolism as lipid regulating drugs, such as thiazolidindiones, e.g. rosiglitazone or pioglitazone, and fibrates, e.g. clofibrate or fenofibrate. PPAR β/δ ligands have been found to be modulators also of wound healing, hair growth and particularly inflammatory responses. (see e.g. Barish G D et al. Trends in Endocrinology and Metabolism. 2004; 15: 158-165. Desvergne B et al. Molecular Endocrinology 2004; 18: 1321-1332. Tan NS et al. EMBO Journal 2004; 23: 4211-4221. Genolet R et al. Current Drug Targets—Inflammation and Allergy. 2004; 3: 361-375. Di-Poi N et al. Lipids 2004; 39: 1093-1099. Tan NS et al. Expert Opin. Ther. Targets 2004; 8: 39-48. Di-Poi N et al. Mol Cell Biol 2005; 1696-1712. Nawrocki AR et al. Drug Discovery Today 2005; 10: 1219-1230.)

In the present invention it has now been found that the treatment with a combination of the selected RXR agonists or selected RXR antagonists with any or more of the PPAR ligands, leads to a higher therapeutic effect, either additive or even preferably super-additive/synergistic, than the RXR agonists or RXR antagonists or the PPAR ligands when given as single agent treatment for prevention and treatment of metabolic and cardiovascular diseases.

### DETAILED DESCRIPTION OF THE INVENTION

In the subsequent detailed specification, whereever the term USE is employed, this refers to the use of one or more retinoid agonists and/or antagonists comprising retinoids with (especially selective) Retinoid X Receptor agonistic and/or antagonistic activity alone or in combination with one or more peroxisome proliferator activated receptor (PPAR α, β/δ, γ) ligands for the manufacture of a medicament or combination product for the treatment (this term wherever used including prevention/prophylaxis and/or therapy) of one or more diseases falling under the generic term metabolic syndrome, especially one or more diseases selected from the group consisting of diabetes type II, obesity, dyslipidemia, hypertension, atherosclerosis and other cardiovascular diseases. It relates to the use of the mentioned compounds or combinations for the treatment of any one or more of these diseases, to a method of treatment of one or more of said diseases comprising administering one or more such compounds or combinations to a warm-blooded animal, especially a human patient, especially to a patient in need of such treatment in a dose that is effective in said treatment, to one or more such compounds or combinations for use in the treatment of one or more of said diseases and/or to a pharmaceutical composition or combination product comprising one or more such compounds or combinations preferably in an amount effective in said treatment, if not indicated otherwise. Especially, such USE comprises a manufacture of a pharmaceutical composition or a combination product for a direct administration to a subject (especially a human patient) expected to be developing or especially already having one or more diseases selected from the group consisting of diabetes type II (non-insulin dependent diabetes mellitus (NIDDM)), obesity, dyslipidemia, hypertension, atherosclerosis and other cardiovascular diseases with manifestation especially in peripheral arteries, in the coronary arteries, in arteries of the brain, the kidney, the eyes, the pancreas, in the form of occlusion, thrombosis and embolism. Wherever “metabolic syndrome” is mentioned, this is intended to include also one or more complications associated with one or more the diseases falling under this term, in particular cardiovascular complications and atherosclerosis.

In the scope and disclosure of the present invention the terms “RXR agonists” and “RXR antagonists” are used for retinoids with RXR selective agonistic or antagonistic activity. The term “PPAR ligands” is used for ligands to PPAR α, β/δ or γ, with agonistic or antagonistic activity.

The present invention relates in particular to the USE of any one or more of the following compounds listed in Table 1, RXR agonists (1a) and RXR antagonists (1b), in Table 2 as well as those listed in Table 3, PPAR ligands, preferably with the definite exception of the compounds 2 and 3, more preferably with the exception of compounds 2, 3, 5, 9, 12, 14 and 24. Most preferably, the invention relates to the USE of the compounds 1, 15 and/or 21.

TABLE 1a
RXR Agonists

| Compound | Chemical Name |
|---|---|
| Compound 1 | (2E,4E)-3-methyl-5-[2-[(E)-2-(2,6,6-trimethyl-1-cyclohexen-1-yl)ethenyl]-1-cyclohepten-1-yl]-2,4-pentadienoic |
| | acid |
| Compound 2 | 4-[1-(5,6,7,8-tetrahydro-3,5,5,8,8-pentamethyl-2-naphthalenyl)ethenyl]benzoic acid |
| Compound 3 | 6-[1-(3,5,5,8,8-Pentamethyl-5,6,7,8-tetrahydro-naphthalen-2yl)-cyclopropyl]-nicotinic acid |
| Compound 4 | (2E,4E)-3-methyl-5-[(1RS,2RS)-2-(5,5,8,8-tetramethyl-5,6,7,8-tetrahydro-naphthalen-2-yl)-cyclopropyl]-penta- |
| | 2,4-dienoic acid |
| Compound 5 | (2E,4E)-3-Methyl-5-[2-[(E)-2-(2,6,6-trimethyl-1-cyclohexen-1-yl)ethenyl]-3-thienyl]-2,4-pentadienoic acid |
| Compound 6 | (2E,4E)-3-Methyl-5-[2-(5,5,8,8-tetramethyl-5,6,7,8-tetrahydro-naphthalen-2-yl)-cyclohex-1-enyl]-penta-2,4- |
| | dienoic acid |
| Compound 7 | (2E,4E)-3-methyl-5-[2-(5,5,8,8-tetramethyl-5,6,7,8-tetrahydro-naphthalen-2-yl)-phenyl]-penta-2,4-dienoic acid |
| Compound 8 | (2E,4E)-3-Methyl-4-(4′,4′,5′,7′,7′-pentamethyl-1′,3′,4′,5′,6′,7′-hexahydro-spiro[cyclopent-4-ene-1,2′-[2H]inden]-3- |
| | ylidene)-but-2-enoic acid |
| Compound 9 | (2E,4E)-3-Methyl-5-[3-(5,5,8,8-tetramethyl-5,6,7,8-tetrahydro-naphthalen-2-yl)]-thiophen-2-yl]-penta-2,4-dienoic |
| | acid |
| Compound 10 | (2E,4E)-3-Methyl-5-[(1S,2S)-2-(5,5,8,8-tetramethyl-5,6,7,8-tetrahydro-naphthalen-2-yl)-cyclopropyl]-penta-2,4- |
| | dienoic acid |
| Compound 11 | (2E,4E,6Z)-(RS)-7-(3-Ethyl-5-methyl-5,6,7,8-tetrahydro-naphthalen-2-yl)-3-methyl-octa-2,4,6-trienoic acid |
| Compound 12 | (2E,4E)-3-Methyl-5-[2-(5,5,8,8-tetramethyl-5,6,7,8-tetrahydro-naphthalen-2-yl)-cyclopent-1-enyl]-penta-2,4- |
| | dienoic acid |
| Compound 13 | (2E,4E,6Z)-3-Methyl-7-(3,5,5,-trimethyl-6,7,8,9-tetrahydro-5H-benzocyclohepten-2-yl)-octa-2,4,6-trienoic acid |
| Compound 14 | (2E,4E)-3-Methyl-5-[2-[(E)-2-(2,6,6-trimethyl-1-cyclohexen-1-yl)ethenyl]-1-cyclopenten-1-yl]-2,4-pentadienoic |
| | acid |

TABLE 1b
RXR Antagonists

| Compound | Chemical Name |
|---|---|
| Compound 15 | (2E,4E,6Z)-7-[2-butoxy-3,5-bis(1,1-dimethylethyl)phenyl]-3-methyl-2,4,6-octatrienoic acid |
| Compound 16 | (2E,4E)-(1RS,2RS)-5-[2-(3,5-Di-tert-butyl-2-butoxy-phenyl)-cyclopropyl]-3-methyl-penta-2,4-dienoic acid |
| Compound 17 | (2E,4E)-(1RS,2RS)-5-[2-(3,5-Di-tert-butyl-2-ethoxy-phenyl)-cyclopropyl]-3-methyl-penta-2,4-dienoic acid |
| Compound 18 | (2E,4E)-3-Methyl-5-[2,6,6-trimethyl-cyclohex-1-enylethynyl)-cyclohept-1-enyl]-penta-2,4-dienoic acid |
| Compound 19 | (2E,4E,6Z)-7-(2-Heptyloxy-3,5-diisopropyl-phenyl)-3-methyl-octa-2,4,6-trienoic acid |
| Compound 20 | (2E,4E)-5-[2-(3,5-Di-tert-butyl-2-propoxy-phenyl)-cyclopropyl]-3-methyl-penta-2,4-dienoic acid |
| Compound 21 | (2E,4E,6Z)-7-[3,5-Bis(1,1-dimethylethyl)-2-ethoxyphenyl]-3-methyl-2,4,6-octatrienoic acid/ethyl ester |
| Compound 22 | (2E,4E)-3-Methyl-5-[(1RS,2RS)-2-(5,5,8,8-tetramethyl-3-propoxy-5,6,7,8-tetrahydro-naphthalen-2-yl)- |
| | cyclopropyl]-penta-2,4-dienoic acid |
| Compound 23 | (2E,4E,6Z)-3-Methyl-7-(5,5,8,8-tetramethyl-3-propoxy-5,6,7,8-tetrahydro-naphthalen-2-yl)-octa-2,4,6-trienoic |
| | acid |
| Compound 24 | (2E,4E,6Z)-7-(5,5-Diethyl-8,8-dimethyl-5,6,7,8-tetrahydro-naphthalen-2-yl)-3-methyl-octa-2,4,6-trienoic acid |

TABLE 2

| | | |
|---|---|---|
| RXRα Agonists | | Transactivation |
| (Type Compound 1) | | |
| RXRα Homodimer | | + |
| PPARγ/RXRα | | + |
| Heterodimer | | |
| Compound 1 | 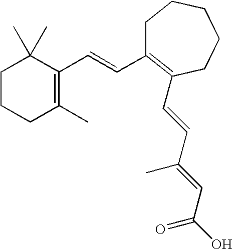 | |
| Compound 2 | 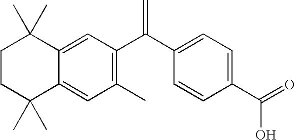 | |
| Compound 3 | 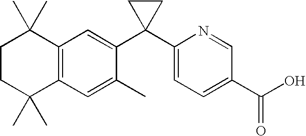 | |
| Compound 4 | 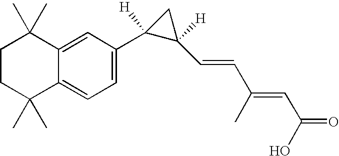 | |
| Compound 5 | 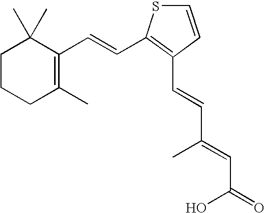 | |
| Compound 6 | 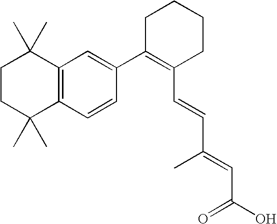 | |
| Compound 7 | 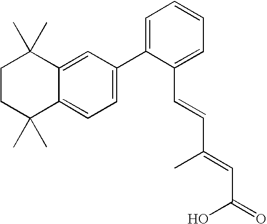 | |
| Compound 8 | 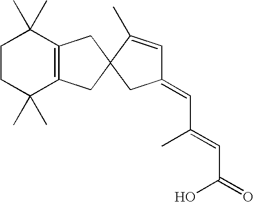 | |
| Compound 9 | 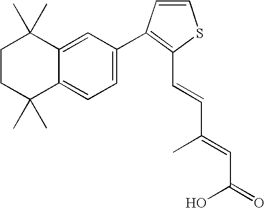 | |
| Compound 10 | 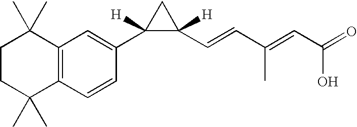 | |
| Compound 11 | 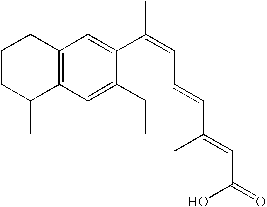 | |
| Compound 12 | 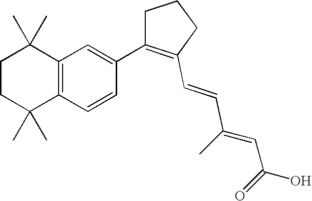 | |
| Compound 13 | 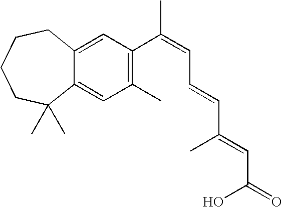 | |
| Compound 14 | 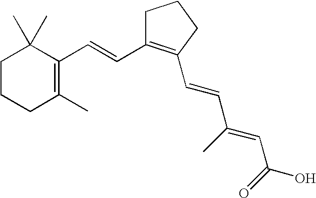 | |
| RXRα Antagonists | | Transactivation |
| (Type Compound 15) | | |
| RXRα Homodimer | | − |
| PPARγ/RXRα Heterodimer | | − |
| Compound 15 | 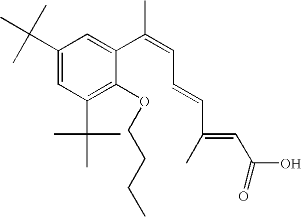 | |
| Compound 16 | 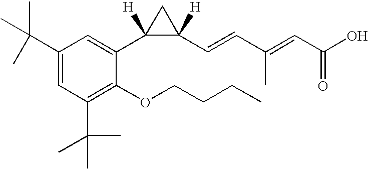 | |
| Compound 17 | 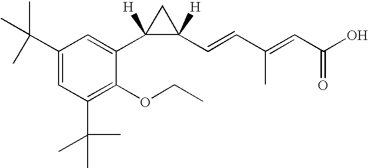 | |
| Compound 18 | 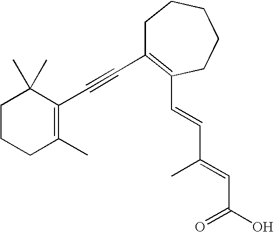 | |
| Compound 19 | 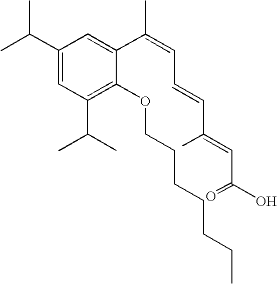 | |
| Compound 20 | 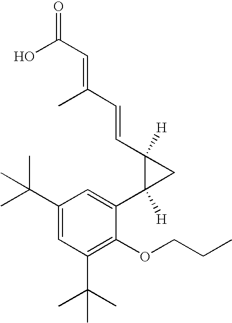 | |
| RXRα Antagonists | | Transactivation |
| (Type Compound 21) | | |
| RXRα Homodimer | | − |
| PPARγ/RXRα Heterodimer | | + |
| Compound 21 | 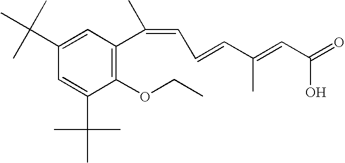 | |
| Compound 22 | 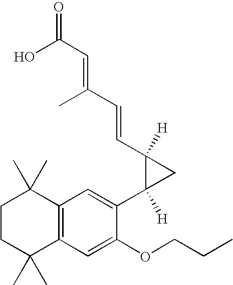 | |
| Compound 23 | 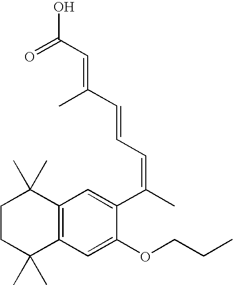 | |
| Compound 24 | 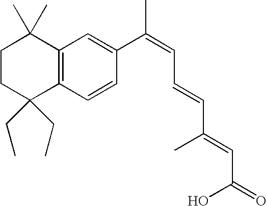 | |

TABLE 3
PPAR Ligands

| | |
|---|---|
| PPARα agonists | e.g. Clofibrate |
| PPARβ/δ agonists | |
| PPARγ agonists | e.g. Rosiglitazone |

Where reference is made to a RXR agonist or RXR antagonist within the present disclosure, this preferably refers to the compounds 1 to 24 (preferably to those compounds which are of USE preferably as defined above and below), an ester or an amide thereof, each in free form and/or in the form of a pharmaceutically acceptable salt (=“a pharmaceutically acceptable amide, ester and/or salt thereof”).

In accordance with this invention, it has been found that administration of a RXR agonist and a RXR antagonist as single agents or in combination with a PPAR ligand, are efficacious in treating warm-blooded animals, especially human patients, with metabolic and cardiovascular diseases.

Preferably, the diseases to be treated with one or more RXR agonists and/or RXR antagonists alone or in combination with one or more PPAR ligands are selected from one or more of the following diseases:
1. Diabetes type II, Non insulin dependent diabetes mellitus (NIDDM)
   This includes patients with manifest diabetes as well as persons with a trend to develop diabetes, e.g. persons with a pathological glucose tolerance test.
   It includes not only the treatment of the pathological metabolic disturbance of diabetes, but includes also all the complications accompanying diabetes or being consequences of the diabetic metabolism or being linked with a high risk of developing atherosclerosis.
   It especially relates to the treatment of one or more up to all the other diseases belonging to the so-called Metabolic Syndrome, manifest as obesity, dyslipidemia, hypertension, atherosclerosis and/or other cardiovascular diseases. It also relates to the (especially oral and/or topical) treatment of chronic wounds, in particular diabetic leg ulcers, of diabetic retinopathy, and/or the reduced defence mechanism of diabetic patients against bacterial, viral and fungal infections.
2. Obesity
   Obesity is part of the metabolic syndrome, as a predisposing factor for diabetes as well as an accompanying disease.
   The indication for treatment of obesity is mainly dependent on the degree of obesity, determined e.g. by the body mass index and on the localization of obesity in particular abdominal obesity. Persons with overweight or a body mass index of 25 or more are treated. Persons with real obesity and in particular abdominal obesity or with a body mass index of 30 or more, profit from treatment especially.
3. Dyslipidemia
   Dyslipidemia is part of the metabolic syndrome and is treated when total cholesterol is higher than 6.5 mmol/l, when LDL and VLDL are increased and when HDL is below 1 mmol/l or when the ratio or quotient of cholesterol/HDL mmol/mmol is higher than 5.
   Treatment is useful when serum triglycerides are higher than 2 mmol/l.
4. Hypertension
   Hypertension is also part of the metabolic syndrome and has to be treated, especially when diabetes and/or obesity and/or dyslipidemia are present in the same person.
5. Atherosclerosis and other cardiovascular diseases
   A series of cardiovascular diseases are successfully treated that include degenerative and inflammatory processes involving alterations of the arterial wall leading to stenosis, narrowing by plaques followed by thrombotic processes and finally in embolism or a complete occlusion of arteries of different diameters. This affects the arteries of many organs of the body, such as arteries of the peripheral limbs, particularly the legs, the coronary arteries leading finally to cardiac infarction, the cerebral arteries leading to stroke or apoplectic fit on the basis of thrombotic occlusion or a hemorrhage.
   Furthermore, it can include alteration of the vessels of the kidney leading to glomerulosclerosis and diabetic nephropathy.
   Hypertension and diabetes can also lead to alterations of the vessels of the eye, such as diabetic retinopathy, thrombotic occlusion, embolic occlusion and hemorrhage which thus can also be treated according to the present invention.
6. Polyneuropathy
   Polyneuropathy, a rather frequent complication of diabetes type II, can also be treated.

Besides these diseases, also one or more other diseases and complications can fall under the term “metabolic syndrome”, such as especially cardiovascular diseases, in particular atherosclerosis.

The term “treatment” includes preventive (prophylactic) and/or especially therapeutic treatment. The one or more compounds or combinations are preferably administered in an amount effective to treat said disease or diseases, especially to a patient in need of such treatment. For the treatment of the above mentioned diseases, one or more of the active compounds, i.e. RXR agonists, RXR antagonists, a pharmaceutically acceptable ester or amide thereof, or a pharmaceutically acceptable salt of these, alone or (in the case of combinations between the RXR (ant)agonist(s) and PPAR ligand(s)) with simultaneous or sequential administration of PPAR ligands are administered either systemically or topically. Preferably, the compound, compounds or combinations are administered as a composition containing, beyond the active compound or compounds, one or more pharmaceutically acceptable carrier materials or diluents compatible with said active compound. In preparing such composition, any conventional pharmaceutically acceptable carrier can be utilized.

When the drug is administered orally, it is generally administered at regular intervals, for example conveniently at mealtimes or once daily. Based on information from toxicological studies (see also below), the RXR agonists and RXR antagonists are effective in doses which show no or only mild side effects when given orally or when given topically. Therefore, oral or topical administration of the active compound is generally preferred. However, oral combined with topical administration may also be used advantageously, for example for treating diseases of the skin e.g. chronic wounds or diabetic leg ulcers, as well as for treating diseases of mucous membranes and of other tissues and organs, e.g. of the eyes, such as diabetic retinopathy.

“Alone”, when mentioned in connection with RXR antagonists or agonists, does not necessarily mean that only one such compound is used, but rather that only such compounds are used without combination with a PPAR ligand. The opposite to this “alone” is thus that a combination with one or more PPAR ligands is meant.

In the treatment of the above-mentioned diseases, RXR agonists and RXR antagonists, especially when administered orally, do not or only slightly induce the adverse events belonging to the toxic syndrome of hypervitaminosis A, such as mucocutaneous, musculoskeletal, neurologic manifestations and elevation of transaminases. On the contrary, regarding triglycerides and cholesterol even beneficial effects can be found as described below. In addition, they are less teratogenic in contrast to the RAR receptor agonistic retinoids clinically useful in the treatment of dermatological and oncological diseases, such as all-trans retinoic acid (tretinoin), 13-cis retinoic acid (isotretinoin), etretinate and acitretin, which have a very high risk of teratogenicity.

In the treatment of the above mentioned diseases (especially under 1. to 5.) RXR agonists and RXR antagonists, their pharmaceutically acceptable salts or pharmaceutically acceptable esters or amides thereof, can be used alone or in combination with PPAR ligands. The RXR agonist(s) and/or antagonist(s) and the PPAR ligand(s) (referred to as active ingredients in the description of pharmaceutical formulations and dosing recommendations hereinafter) can also be combined with antibacterial, antifungal or antiviral agents administered topically and/or systemically.

If used in combination with other substances, one or more RXR agonists and/or RXR antagonists and one or more PPAR ligands can be administered separately in separate pharmaceutical formulations, or they can be incorporated in effective amounts into one pharmaceutical composition, or especially they can form a kit of parts the components of which may be administered at separate (=sequentially) or overlapping time periods and/or at the same time (simultaneously), especially in such a way that the beneficial effects are overlapping or even enhancing each other in an additive or preferably even synergistic way. The term “combination product” as used herein especially refers to fixed combinations of two or more of the active ingredients such kits of parts or to products comprising the active ingredients in separate formulations, however with an indication that they are to be or can be used in combination with each other.

The aforementioned RXR agonists and/or antagonists and the PPAR ligands are especially useful preferably in pharmaceutically acceptable oral or topical formulations. These pharmaceutical compositions comprise an active compound in association with a compatible pharmaceutically acceptable carrier material.

Any one or more conventional carrier materials suitable for oral administration can be used. Suitable carriers include water, gelatine, gum arabic, lactose, starch, magnesium stearate, talc, vegetable oils, polyalkylene-glycols, petroleum jelly and the like. Furthermore, the pharmaceutically active preparations may contain other pharmaceutically active agents. Additionally, additives such as flavouring agents, preservatives, complexing agents, pigments, dyes. Any one or more further additives selected from the groups consisting of stabilizers, tensides, emulsifying agents, wetting agents, solubilizers, buffers and the like may be added in accordance with acceptable practices of pharmaceutical compounding. Appropriate carrier materials (also for other formulations described herein) can, for example, be deduced from the pharmacopoeias, e.g. the European Pharmacopoeia (Ph.Eur.), the German DAB or the US pharmacopoeia, especially in their last edition before the filing date of the present invention, respectively, which are included by reference in this regard herewith.

The pharmaceutical preparations can be made up in any conventional form including inter alia: (a) a solid form for oral administration such as tablets, capsules (e.g. hard or soft gelatine capsules), pills, sachets, powders, granules, and the like; (b) preparations for topical administrations such as solutions, suspensions, ointment, creams, hydrogels, lipogels, micronized powders, sprays, aerosols and the like. The pharmaceutical preparations may be sterilized and/or may contain adjuvants such as preservatives, stabilizers, wetting agents, emulsifiers, salts for varying the osmotic pressure and/or buffers.

For topical administration to the skin or mucous membranes the active compound(s) is or are preferably prepared as ointments, tinctures, creams, gels. solution, lotions; nasal sprays; aerosols and dry powder for inhalation; suspensions, shampoos, hair soaps, perfumes and the like. In fact, any conventional composition can be utilized in this invention. Among the preferred methods of applying the composition containing the agents of this invention is in the form of an ointment, gel, cream, lotion; nasal spray, aerosol or dry powder for inhalation. The pharmaceutical preparation for topical administration to the skin can be prepared by mixing the aforementioned active ingredient with non-toxic, therapeutically inert, solid or liquid carriers customarily used in such preparations. These preparations preferably comprise 0.1 to 20 percent by weight, especially 0.1 to 5.0 percent by weight, preferably 0.3 to 2.0 percent by weight, of the active compound, based on the total weight of the composition.

In preparing the topical preparations described above, additives such as preservatives, thickeners, perfumes and the like customary in the art of pharmaceutical compounding of topical preparation can be used. In addition, conventional antioxidants or mixtures of conventional antioxidants can be incorporated into the topical preparations containing the aforementioned active agent. Among the conventional antioxidants which can be utilized in these preparations are included N-methyl-α-tocopherolamine, tocopherols, butylated hydroxyanisole, butylated hydroxytoluene, ethoxyquin and the like. Cream-base pharmaceutical formulations containing the active agent, used in accordance with this invention, are composed of aqueous emulsions containing a fatty acid alcohol, semi-solid petroleum hydrocarbon, ethylene glycol and an emulsifying agent.

Ointment formulations containing the active agent in accordance with this invention, for example, comprise admixtures of a semi-solid petroleum hydrocarbon with a solvent dispersion of the active material. Cream compositions containing the active ingredient for use in this invention preferably comprise emulsions formed from a water phase of a humectant, a viscosity stabilizer and water, an oil phase of a fatty acid alcohol, a semi-solid petroleum hydrocarbon and an emulsifying agent and a phase containing the active agent dispersed in a aqueous stabilizer-buffer solution. Stabilizers may be added to the topical preparation. Any conventional stabilizer can be utilized in accordance with this invention. These fatty acid alcohol components function as a stabilizer. These fatty acid alcohol components are derived from the reduction of a long-chain saturated fatty acid containing at least 14 carbon atoms. Also, conventional perfumes and lotions generally utilized in topical preparation for the hair can be utilized in accordance with this invention. Furthermore, if desired, conventional emulsifying agents can be utilized in the topical preparations of this invention. Alternatively gels can be used utilising standard gel carriers.

Examples for a possible preferred oral dosage form for RXR agonists and/or RXR antagonists comprise tablets, pills, sachets, or capsules of hard or soft gelatine, methylcellulose or of another suitable material easily dissolved in the digestive tract. Each unit dosage form (e.g. tablet, pill, sachet or capsule) can preferably contain from about 5 to about 2000 mg, especially 10 to about 500 mg, more preferably from about 20 to about 200 mg, of active ingredients.

The (especially oral) dosages contemplated in accordance with the present invention may vary in accordance with the needs of the individual patient (e.g. the condition of the patient, the size, the age, possible interferences with other therapeutic measures and the like) as determined by the prescribing physician. Generally, however, a daily dosage of from 0.1 to 50, especially 0.2 to 20 mg per kg of body weight, preferably 0.5 to 10 mg, and most preferably from about 1 mg to about 3 mg per kg of body weight of the patient and per active compound is administered. This dosage may be administered according to any dosage schedule determined by the physician in accordance with the requirements of the patient. For example, an adult patient may be administered from 7 to 3500 mg, e.g. from 14 to 1400 mg, especially from 35 to 700 mg, more preferably from 70 to 210 mg of an RXR agonist or of an RXR antagonist daily in one or more, e.g. up to three, partial doses a day.

The dosage for treatment typically depends on the route of administration, the age, weight and disease condition of the individual. Suitable dosage forms are known in the art or can be easily obtained in a manner known per se. Formulations of solutions, suspension, lotions, gels, creams, sprays; aerosols and dry powder for inhalation, hard or soft gelatine capsules, pills, tablets and sachets that are particularly suitable in the scope of the present invention can be easily adjusted in accordance with the above teaching and the general knowledge in the art.

The route of administration, the pharmaceutical formulation, the dosage, efficacy, and side effects in the treatment of the various therapeutic indications of metabolic and cardiovascular diseases with PPAR ligands are well known. They are described in detail in compendia of pharmaceutical products which have been officially introduced onto the market with product information for the doctor and/or for the patient. This especially relates to, regarding the PPARα ligands, the fibrates e.g. Clofibrate or Fenofibrate, regarding the PPAR γ ligands, the glitazones e.g. Rosiglitazone or Pioglitazone, and the PPAR β/δ ligands. The dosages may lie in the range from 1 to 3000 mg per patient and day for adult persons, e.g. in the case of rosiglitazone in the range from 1 to 10 mg per day, e.g. from 4 to 8 mg per day, in the case of pioglitazone in the range from 10 to 100 mg per day, e.g. from 30 to 45 mg per day, in the case of clofibrate in the range from 500 to 3000 mg per day, e.g. from 1000 to 2000 mg per day, or in the case of fenofibrate from 100 to 1000 mg per day, e.g. at about 400 mg per day.

The present invention deals with the successful treatment of the above mentioned metabolic, cardiovascular and neurological diseases and their accompanying clinical complications by compounds listed in Tables 1 and 2, preferably with the definite exception of compounds 2 and 3, and more preferably with the exception of compounds 2, 3, 5, 9, 12, 14 and 24, where USE of a compound selected from RXR agonist compounds 1, 4, 6, 7, 8, 10, 11 and/or 13 and/or the RXR antagonists 15, 16, 17, 18, 19, 20, 21, 22 and/or 23 is more preferred, while USE of a compound selected from compounds 1, 15 and/or 21 is most preferred; in the case of combinations with one or more PPAR ligands especially a compound mentioned in Table 3 (preferably a glitazone or fibrate mentioned therein), each administered as single agent or in one or more of the possible combinations with each other, which can be administered preferably topically or more preferably orally.

The classical, conventional and most frequently used drugs for treatment of all the mentioned diseases include insulin, sulfonylureas and biguanides for treatment of diabetes mellitus and also all the drugs for treatment of obesity, lipid disorders and cardiovascular diseases, such as orlistat, lipid lowering agents, statins, antihypertensives, β-receptor blockers, calcium antagonists and drugs for congestive heart failure. The treatment with these drugs were more recently complemented by drugs belonging to the group of peroxisome proliferator activated receptor (PPAR α, β/δ and γ) ligands, such as fibrates, glitazones and others.

All these drugs have been proven to be of great importance for the successful treatment of these diseases. In spite of the good therapeutic results with the conventional drugs there is still an urgent medical need for better drugs that exert a still higher efficacy and/or induce a lower toxicity i.e. less side effects and less adverse events.

It is the purpose of the present invention to show that compounds listed in Tables 1 and 2, with the exception of compounds 2 and 3, preferably also with the exception of other compounds to be preferably excepted as mentioned above, given as single agents or particularly in combination with one or more PPAR ligands, especially selected from the compounds listed in Table 3, quite unexpectedly, possess a better quotient between efficacy and toxicity, or have a more favorable ratio of efficacy to toxic side effects, than the conventional drugs used for therapy of the metabolic or cardiovascular diseases (also including dermatological or oncological diseases for which prior art compounds are known to be of use).

In the case of retinoids, administered as single agents or in combination with PPAR ligands, two sorts of side effects play a dominant role.

When applied topically to the skin, irritation or inflammation of the skin is induced, which is a major handicap of this kind of administration. The compounds listed in Tables 1 and 2 of the present invention do not, or only slightly induce irritation or inflammation of the skin when administered topically to the skin of animals or humans.

Furthermore, when these compounds with the exception of compounds 2 and 3 (and preferably other compounds that fall under the preferred exceptions given above) are administered orally or parenterally they do not or only slightly induce the adverse events belonging to the toxic syndrome of hypervitaminosis A including mucocutaneous manifestations, such as dry skin, cheilitis, flush and conjunctivitis, musculoskeletal symptoms, such as myalgia, osteopenia, osteoporosis, bone factures or hyperostosis; neurological manifestations, such as headache, as well as abnormalities of biochemical parameters, such as elevation of transaminases, elevation of triglycerides, elevation of total cholesterol, increase of low density lipoprotein (LDL) and very low density lipoprotein (VLDL) and decrease of high density lipoprotein (HDL). On the contrary, the preferred compounds (those remaining after removal of compounds that are preferably excepted)

In experimental investigations on db/db mice with RXR agonists it is shown below that compound 2, in contrast to compounds 1 and 4 to 14, increases blood levels of triglycerides and decreases HDL-cholesterol. This is in agreement to results in clinical trials wherein an increase of serum triglycerides, an increase of low density lipoprotein (LDL) and a decrease of high density lipoprotein (HDL-cholesterol) is observed (Miller VA et al. J Clin Oncol 1997; 15: 790-795. Rizvi NA et al. Clin. Cancer Res 1999; 5: 1658-1664). This is similar with compound 3.

Since an increase of triglycerides, an increase of LDL and a decrease of HDL-cholesterol are high risk factors for development of atherosclerosis, the use of agents like compounds 2 and 3 is not desirable for treatment of metabolic syndrome related diseases, e.g. diabetes type II, obesity, dyslipidemia and cardiovascular diseases.

The RXR agonists, RXR antagonists and PPAR ligands mentioned above and below can be provided or used in a USE according to the invention in free form or in the form of a pharmaceutically acceptable salt (which can be present if salt-forming groups are present), or in the form of a pharmaceutically acceptable amide (which can be present if groups that can form amides are present such as COOH, NH or NH₂) and/or ester (which can be present if groups that can form esters are present such as COOH, OH, SO₃H), where also the amides and ester can be present in the form of a pharmaceutically acceptable salt thereof (which can be present if salt-forming groups are present), can be used. Where reference is made to one or more RXR agonists, RXR antagonists or PPAR ligands, this term is always intended to also include these alternative forms to the free form, even if not explicitly mentioned, if not mentioned otherwise, and in addition solvates and specific crystal forms.

The expression “pharmaceutically acceptable salts” includes any salt chemically permissible in the art for retinoid agonists or antagonists that bear at least one salt-forming group, e.g. an acidic group, such as carboxyl or sulfonyl, and that can be administered to warm-blooded animals, especially human beings (e.g. patients), for example in a pharmaceutically acceptable composition. Any conventional pharmaceutically acceptable salt of retinoid agonists or antagonists can be utilised. Among the conventional salts which can be made use of, there are the base salts included, for example, alkali metal salts such as the sodium or potassium salt, alkaline earth metal salts such as the calcium or magnesium salt, and ammonium or alkyl ammonium salts. Where basic groups are present, these can be in the form of their acid addition salts, e.g. with organic acids, e.g. in the form of the acetate, methylsulfonate (mesylate) or fumarate, or inorganic acids, e.g. in the form of the sulphate, chloride or bromide. even show beneficial effects regarding triglyceride level and cholesterol.

It is particularly unexpected that the oral administration of the compounds mentioned in Tables 1 and 2, with the exception of compounds 2 and 3, preferably also with the exception of other compounds to be preferably excepted as mentioned above, do not or only slightly, induce the signs and symptoms of the toxic hypervitaminosis A syndrome. This is in sharp contrast to all the retinoids on the market for treatment of dermatological and oncological diseases which induce always the signs and symptoms of the hypervitaminosis A syndrome, when given in higher doses or for a prolonged time.

The finding that especially the compounds listed in Tables 1 and 2, with exception of compounds 2 and 3, preferably also with the exception of other compounds to be preferably excepted as mentioned above, are useful in the treatment of metabolic and cardiovascular diseases is especially unexpected since particularly the compounds 1 and 4 to 14 and compounds 15 to 24, more particularly those not excepted above in preferred embodiments of the invention, most preferably the RXR agonist compounds 1, 4, 6, 7, 8, 10, 11 and/or 13 and/or the RXR antagonists 15, 16, 17, 18, 19, 20, 21, 22 and/or 23—in contrast to other retinoids, in particular those with RAR agonistic activity—do not, or only slightly induce irritation or inflammation, when administered topically and do not or only slightly induce the signs and symptoms of the toxic hypervitaminosis A syndrome. Toxicological data for topical and oral administration of compounds 1 and 15 are given in Examples 1 and 2.

### EXAMPLE 1

### Toxicology of Compound 1 (see Table 1)

### Topical Use

Topical administration of RXR agonist compound 1 to the skin of mice and rats in concentrations up to 2.5% does not lead to irritation or inflammation of the skin.

Compound 1, in contrast to retinoids with RAR agonistic activity and also in contrast to certain RXR agonists with even an only low RAR agonistic activity, does not irritate the skin. This is due to the fact that compound 1 has a binding affinity which is highly selective to the RXR receptor compared to affinity to the RAR receptor.

### Clinical Trial

In a human volunteer, compound 1 in a 1% concentration administered daily to the skin for 2 weeks does not induce any irritation or inflammation of the skin.

### Oral Use

Oral administration of the RXR agonist compound 1 is tested by daily oral gavage in a 2 weeks toxicology study in NMRI mice. The compound is very well tolerated orally up to 400 mg/kg/day. Intraperitoneally the compound is well tolerated at the dose of 200 mg/kg/day and a retarded body weight increase is observed at 400 mg/kg/day. In all treated groups, there are no signs or symptoms of the hypervitaminosis A syndrome, such as manifestations on skin, mucous membranes and bones, or biochemical abnormalities, such as elevation of transaminases, triglycerides and total cholesterol. There is even a tendency to a decrease in triglycerides, an increase in HDL-cholesterol and a decrease in LDL-cholesterol. This signifies a favourable effect on the lipid profile, which is important in therapy of diabetes and lipid disorders.

Compound 1 and analogs, in contrast to certain other RXR agonists (compounds 2 and 3) do not induce adverse events. This is explained by the fact that compound 1 and analogs have a highly selective binding affinity to the RXR receptor, with no binding affinity to and no activation of the RAR receptors. Even a low binding affinity to the RAR receptor and its activation, can lead to signs and symptoms of hypervitaminosis A, e.g. increase of triglycerides and cholesterol. Therefore, compound 1 and analogs do e.g. not increase triglycerides, whereas other RXR agonists such e.g. compounds 2 and 3 increase triglycerides. Other properties of the compounds, not yet clearly defined, may also be responsible for or contribute to the induction of undesired side effects.

### EXAMPLE 2

### Toxicology of Compound 15 (see Table 1)

### Topical Use

Topical administration of the RXR antagonist compound 15 to the skin of mice and rats in concentrations up to 2.5% does not lead to irritation or inflammation of the skin.

### Clinical Trial

In a human volunteer compound 15 in a 1% concentration administered daily to the skin for 2 weeks does not induce any irritation or inflammation of the skin.

### Oral Use

In a 4-weeks toxicology study in rats, compound 15 is administered by oral gavage in daily doses of 50, 250, 500, eventually increased to 750 mg/kg. All doses are well tolerated with the exception of patchy alopecia. This symptom of hair loss is observed, dose-dependently, with all doses. Clinical pathology is limited to minor disturbances.

Histopathology revealed only some changes in the 500/750 mg/kg dose group, e.g. on seminiferous tubules, germ cells, and in the cortical zone of adrenal glands. No abnormalities in haematology, no abnormalities in clinical chemistry. No increase in transaminases, triglycerides, and cholesterol, known as side effect of many retinoids.

In a 2-weeks toxicity study in normal mice with daily intraperitoneal administration of compound 15, doses up to 400 mg/kg are well tolerated. There are no signs or symptoms of hypervitaminosis A on skin, mucous membranes or bones with the exception of hair loss. Alopecia is dose dependent.

As mentioned above, the ratio of binding affinity to RAR and RXR and activation of RAR and RXR play a decisive role, whether retinoids induce the signs and symptoms of the hypervitaminosis A syndrome or not, this latter being by far the most important factor in causing undesired side effects and adverse events. It is known that all the retinoids being useful in treatment of dermatological and oncological diseases, such as all-trans retinoic acid (tretinoin), 13-cis retinoic acid (isotretinoin) and the aromatic retinoids etretinate and acitretine induce the hypervitaminosis A syndrome when given in higher doses or for a prolonged period of time. All these retinoids bind and activate RAR receptors. For demonstrating the relation between binding affinity to and activation of RAR and RXR receptors the data of the corresponding assays are given for the compounds 1 to 24 listed in Tables 1, 2 and 3, and are given in Example 3. In contrast, to the above mentioned retinoids, the majority of these compounds 1-24 do not activate RARs or only to a low degree and do therefore not induce the hypervitaminosis A syndrome.

It is, however, to be emphasized that also compounds with some RAR affinity may still be useful in the USE according to the present invention.

### Pharmacokinetics (PK):

Single dose administration: Compound 15 is administered orally as a microsuspension in oil. When measured one hour after oral administration, plasma levels are 480 ng/ml with 20 mg/kg and 2755 ng/ml with 100 mg/kg. There is a dose proportional increase in plasma levels of compound 15 within this dose range. However, doses higher than 100 mg/kg did not lead to a further increase in plasma levels. By comparing intranvenous and oral PK data of compound 15, oral bioavailability at the mentioned low doses is 40%.

Daily administration of compound 15 by oral gavage for 14 days in mice: Compound 15 is administered daily by oral gavage as a microsuspension in oil in doses of 30 to 400 mg/kg/day for 14 days in mice. Compound 15 is rapidly absorbed after oral administration with Tmax between 0.5 and 2 h. The systemic exposure to compound 15, determined by Cmax and AUC last, increases almost dose proportionally between 30 and 100 mg/kg/day. Cmax of 4443 ng/ml with 30 mg/kg increases to 8916 ng/ml with 100 mg/kg, and AUC of 13180 ng×h/ml with 30 mg/kg increases to 25715 ng×h/ml with 100 mg/kg. Higher oral doses do not lead to higher Cmax and AUC values.

Daily administration of compound 15 by oral gavage for 4 weeks in rats: Compound 15 is administered daily by oral gavage as an oily suspension in doses of 50, 250, 500 and 750 mg/kg/day for 4 weeks in rats. Plasma samples are taken 2 hours post dosing on days 1, 22 and 26 and analysed by a gradient HPLC method with UV detection. With the 50 mg/kg/day dosage, plasma levels increase from 2205 ng/ml on day 1 to 2235 ng/ml on day 22 and to 2505 ng/ml on day 26. With the 250 mg/kg/day dosage, plasma levels change from 4945 ng/ml on day 1 to 4190 ng/ml on day 22 and to 4530 ng/ml on day 26. Exposure of the rats to compound 15 results in a dose-proportional way in the doses between 50 and 250 mg/kg/day. However, with 500 and 750 mg/kg/day, plasma levels are not significantly different from those with 250 mg/kg/day.

### EXAMPLE 3

### Retinoid Binding and Activation

### Receptor Binding and Activation

Binding and activation test for retinoid receptors RARs and RXRs are done with the following assays. The compounds 1 to 24 are tested.

### Method

1. Retinoid Binding Assays

The DEF domains of RAR's and the full length RXR's, expressed in E.coli, are used to measure competitive retinoid binding. The radiolabelled ligand is 5 nM [³H] all-trans retinoic acid (for RAR's) and 20 nM [³H] 9-cis retinoic acid (for RXR's), respectively. Aliquots of receptors (crude extracts; 0.2-0.4 pmol) are incubated in presence of increasing concentrations of the unlabelled test compound for 3 hours at room temperature (for buffers and detailed conditions see C. Apfel et al., Proc. Natl. Acad. Sci. USA (1992) 98, 7129-33, C. Apfel et al., J. Biol. Chem. (1995) 270, 30765-72, and P. LeMotte et al., Biochim. Biophys. Acta (1996) 1289, 298-304). Separation of bound from free radioactivity is achieved by charcoal/dextran (RAR's) or by desalting columns (RXR's). Results are given as IC₅₀ (nM), the concentration of test compound leading to 50% inhibition of binding of the labelled retinoic acid.
2. Retinoid Activation Assays

### RAR's

Chimeric RAR cDNAs are used for transfection which contain the DNA binding region of the estrogen receptor. As reporter system, the SeAp (secreted alkaline phosphatase) gene under control of the vitellogenin estrogen response element fused to the herpes simplex thymidinkinase promoter (vit-TK-SeAP) is used. The galactosidase expression vector pCH110 serves to correct for variation in transfection efficiency. COS-1 cells are transiently transfected, 24 h before the experiment, by the DEAE-dextran method. The transfected cells are 18 h later replated in 96 well plates and thereafter incubated for 36-48 hours with the test retinoid at various concentrations. At the end of the incubation, the cell supernatants are assayed for SeAP activity. Results are given as EC₅₀ (nM), the concentration leading to half-maximal activation.

### RXR's

Full length RXR cDNA is used for transfection. As reporter system, the luciferase gene is used in a construct with three copies of a RXR response element from rat CRBP gene, (formerly RARE from betaRAR gene), fused to alcoholdehydrogenase gene promoter in the plasmid pGL2-basic. Schneider SL-3 cells from Drosophila are transiently transfected 3-4 h before the experiment by the Ca-phosphate-DNA coprecipitation method. The transfected cells are 18 h later replated in 96 well plates and thereafter incubated for 36-48 hours with the test retinoid at various concentrations. At the end of the incubation, the cell supernatants are assayed for luciferase activity. Results are given as EC₅₀ (nM), the concentration leading to half-maximal activation.

### Results

The results of the binding affinity and activation are given for the compounds 1 to 24 in the following Table 4. Most of the compounds do neither bind nor activate RAR receptors, whereas some compounds bind to a certain degree RAR receptors or even activate RAR receptors.

TABLE 4
Retinoids: Binding Affinity to Receptors and Their Activation

| | | Activation | | Binding Affinity | |
|---|---|---|---|---|---|
| | | EC50 (nM) | | IC50 (nM) | |
| | | RAR | RXR | RAR | RXR |
| RXR Agonists | | | | | |
| Compound 1 | α | 10,000 | 1.4 | >10,000 | 110 |
| | β | 10,000 | | >10,000 | |
| | γ | 10,000 | 10,000 | >10,000 | 150 |
| Compound 2 | α | 685 | 5 | 9500 | 110 |
| | β | 520 | 23 | 9900 | |
| | γ | 930 | 50 | 5100 | 60 |
| Compound 3 | α | 10,000 | 1 | 10,000 | 30 |
| | β | 10,000 | | 10,000 | |
| | γ | 10,000 | | 10,000 | 90 |
| Compound 4 | α | 10,000 | 3.5 | >10,000 | 84 |
| | β | 1200 | | >10,000 | |
| | γ | 1000 | 50 | 7500 | 127 |
| Compound 5 | α | 170 | 0.8 | 1600 | 150 |
| | β | 85 | 50 | 1000 | |
| | γ | 170 | 10,000 | 5300 | 160 |
| Compound 6 | α | 10,000 | 19 | >10,000 | 330 |
| | β | 10,000 | 1850 | >10,000 | |
| | γ | 10,000 | 140 | >10,000 | 240 |
| Compound 7 | α | 10,000 | 6 | >10,000 | 780 |
| | β | 10,000 | 330 | >10,000 | |
| | γ | 10,000 | 400 | 6300 | 970 |
| Compound 8 | α | 10,000 | 2 | >10,000 | 100 |
| | β | 1000 | 21 | >10,000 | |
| | γ | 10,000 | 210 | >10,000 | 100 |
| Compound 9 | α | 2300 | 2.5 | 3400 | 230 |
| | β | 360 | | 3800 | |
| | γ | 890 | 90 | 3600 | 230 |
| Compound 10 | α | 10,000 | 2.6 | 10,000 | 110 |
| | β | 10,000 | 28 | 10,000 | |
| | γ | 10,000 | 65 | 10,000 | 122 |
| Compound 11 | α | 10,000 | 2.1 | 10,000 | 65 |
| | β | 10,000 | 17 | 10,000 | |
| | γ | 10,000 | 37 | 10,000 | 56 |
| Compound 12 | α | 370 | 9 | 3500 | 80 |
| | β | 84 | 60 | 2500 | |
| | γ | 200 | 150 | 6600 | 120 |
| Compound 13 | α | 10,000 | 1.5 | 6500 | 184 |
| | β | 10,000 | 77 | 10,000 | |
| | γ | 10,000 | 400 | 5300 | 147 |
| Compound 14 | α | 150 | 6 | 10,000 | 110 |
| | β | 90 | 270 | 10,000 | |
| | γ | 150 | 50 | 10,000 | 140 |
| Compound 15 | α | 10,000 | 10,000 | 4200 | 30 |
| | β | 10,000 | | >10,000 | |
| | γ | 10,000 | 10,000 | 2700 | 47 |
| RXR Antagonists | | | | | |
| Compound 16 | α | 10,000 | 10,000 | >10,000 | 65 |
| | β | 10,000 | | >10,000 | |
| | γ | 10,000 | 10,000 | >10,000 | 82 |
| Compound 17 | α | 10,000 | 10,000 | >10,000 | 49 |
| | β | 10,000 | | >10,000 | |
| | γ | 10,000 | 10,000 | >10,000 | 69 |
| Compound 18 | α | 10,000 | 10,000 | >10,000 | 95 |
| | β | 10,000 | 10,000 | >10,000 | |
| | γ | 10,000 | 10,000 | >10,000 | 65 |
| Compound 19 | α | 10,000 | 10,000 | 10,000 | 9 |
| | β | 10,000 | | 10,000 | |
| | γ | 10,000 | | 10,000 | 19 |
| Compound 20 | α | 10,000 | 10,000 | 10,000 | 52 |
| | β | | | | |
| | γ | 10,000 | 10,000 | 10,000 | 71 |
| Compound 21 | α | 10,000 | 10,000 | >10,000 | 24 |
| | β | 10,000 | | >10,000 | |
| | γ | 10,000 | 10,000 | >10,000 | 57 |
| Compound 22 | α | 10,000 | >10,000 | 10,000 | 300 |
| | β | 10,000 | >10,000 | 10,000 | |
| | γ | 10,000 | >10,000 | 10,000 | 700 |
| Compound 23 | α | 10,000 | >10,000 | 3100 | 30 |
| | β | 10,000 | >10,000 | 3500 | |
| | γ | 10,000 | >10,000 | 2800 | 68 |
| Compound 24 | α | 54 | 10,000 | 46 | 680 |
| | β | 9.9 | 10,000 | 20 | |
| | γ | 16.1 | 10,000 | 33 | 660 |

Whereas, on the one hand, all retinoids useful in treatment of dermatological and oncological diseases bind and activate RAR's and induce the hypervitaminosis A syndrome in animals and humans, the compounds 1-24 of the present invention, on the other hand, do not or only to a low degree bind and activate RAR's. On the other side, the compounds of the present invention bind, or bind and activate, RXR, depending on whether they possess either agonistic or antagonistic activity.

The relationships between binding affinity and activation of retinoic receptors and the induction of the toxic hypervitaminosis A syndrome is therefore demonstrated. However, other factors may also play a role as will be dealt with in connection with the pharmacological properties of these compounds, based on molecular biology experiments:

Pharmacological investigations have been carried out with model systems—in vivo on animals—which are considered predictive for a successful treatment of metabolic diseases and cardiovascular diseases comprising the therapeutic indications 1 to 6 of the present invention:

### EXAMPLE 4

### Pharmacological Investigations on Usefulness in the Treatment of Metabolic Diseases Falling Under the Metabolic Syndrome

Test compounds: Compounds 1, 2 and 15

### Pharmacology

The animal model system used for the evaluation of compounds useful in the treatment of metabolic diseases and cardiovascular diseases is the db/db mouse with a genetically determined progressive development of diabetes mellitus, with disturbed glucose metabolism and insulin resistance. The following compounds are investigated and compared to placebo (vehicle control).

Compound 1, a pure RXR agonist, having only RXR agonistic activity and no RAR agonistic activity.

Compound 2, an RXR agonist having RXR agonistic activity but possessing also some RAR agonistic activity (partial RXR agonist).

Compound 15, a pure RXR antagonist, having no RAR activity.

### Method

The mentioned 3 compounds are tested in db/db mice during a time when diabetes is in a rapid developing stage. The compounds are administered daily intraperitoneally or orally for 10 days at 3 different doses of 0.3, 3.0 and 30 mg/kg/day. Each dosage group and the vehicle group comprise 7 mice. The suspensions used are prepared with peroxide free arachis oil as vehicle. Blood samples are collected by retro-orbital bleeding at days −4, 0 and 12. The blood levels of glucose, triglycerides, total cholesterol, and HDL-cholesterol are determined.

### Evaluation

For evaluation of efficacy, the difference between the values of glucose, triglycerides, total cholesterol and HDL-cholesterol changing from day 0 to day 12 is determined and expressed in %. The comparison of the data of the various dosage groups of the 3 compounds 1, 2 and 15 with those of the vehicle group allow the quantitative determination of the efficacy of the compounds. Overall evaluation consists in determining the efficacy of the compounds by calculating the whole extent of the effect expressed in %. This comparison includes only the data of the vehicle group and the data of groups treated with 30 mg/kg/day, a dose which is well tolerated. This value is considered as a valuable indicator for the efficacy of the investigated compounds administered in a tolerated dose.

Results (see Tables 5.1, 5.2 and 5.3)

TABLE 5.1
Investigation with db/db mice
Treatment with Compound 1
Dose i.p. (mg/kg/day)

| Compound 1 | D-4 | D o | D 12 | Diff. D o − D 12 | D-4 | D o | D 12 | Diff. D o − D 12 |
|---|---|---|---|---|---|---|---|---|
| | Glucose (mg/dl) | | | | Triglycerides (mmol/l) | | | |
| Vehicle | 146 | 189 | 282 | +49.2% | 1.77 | 1.81 | 2.21 | +22.1% |
| 0.3 | 138 | 207 | 326 | +57.5% | 1.83 | 1.73 | 2.17 | +25.4% |
| 3.0 | 155 | 194 | 273 | +40.7% | 1.85 | 1.83 | 2.11 | +15.3% |
| 30 | 174 | 335 | 206 | −12.3% | 2.13 | 1.7 | 1.71 | +0.6% |
| | Total cholesterol (mmol/l) | | | | HDL-cholesterol (mmol/l) | | | |
| Vehicle | 4.33 | 4.20 | 3.83 | −8.8% | 2.58 | 2.93 | 2.82 | −3.8% |
| 0.3 | 4.26 | 4.27 | 3.67 | −14.1% | 2.65 | 3.78 | 2.69 | −3.2% |
| 3.0 | 4.25 | 4.68 | 3.75 | −19.9% | 2.53 | 3.37 | 2.71 | −19.6% |
| 30 | 4.37 | 4.21 | 4.54 | +7.8% | 2.46 | 2.94 | 3.37 | +14.6% |

TABLE 5.2
Investigation with db/db mice
Treatment with Compound 2
Dose i.p. (mg/kg/day)

| Compound 2 | D-4 | D o | D 12 | Diff. D o − D 12 | D-4 | D o | D 12 | Diff. D o − D 12 |
|---|---|---|---|---|---|---|---|---|
| | Glucose (mg/dl) | | | | Triglycerides (mmol/l) | | | |
| Vehicle | 146 | 189 | 282 | +49.2% | 1.77 | 1.81 | 2.21 | +22.1% |
| 0.3 | 144 | 189 | 291 | +54.0% | 1.65 | 1.98 | 1.90 | −4.0% |
| 3.0 | 169 | 175 | 229 | +30.4% | 1.97 | 2.01 | 1.51 | −24.8% |
| 30 | 128 | 155 | 240 | +54.8% | 1.75 | 1.61 | 2.26 | +40.4% |
| | Total cholesterol (mmol/l) | | | | HDL-cholesterol (mmol/l) | | | |
| Vehicle | 4.33 | 4.20 | 3.83 | −8.8% | 2.58 | 2.93 | 2.82 | −3.8% |
| 0.3 | 3.96 | 3.99 | 3.50 | −12.3% | 2.48 | 2.71 | 2.39 | −11.8% |
| 3.0 | 4.48 | 4.65 | 3.47 | −25.4% | 2.66 | 2.77 | 2.54 | −8.3% |
| 30 | 4.24 | 4.31 | 3.37 | −21.8% | 2.74 | 3.10 | 2.43 | −21.6% |

TABLE 5.3
Investigation with db/db mice
Treatment with Compound 15
Dose i.p. (mg/kg/day)

| Compound 15 | D-4 | D o | D 12 | Diff. D o − D 12 | D-4 | D o | D 12 | Diff. D o − D 12 |
|---|---|---|---|---|---|---|---|---|
| | Glucose (mg/dl) | | | | Triglycerides (mmol/l) | | | |
| Vehicle | 146 | 189 | 282 | +49.2% | 1.77 | 1.81 | 2.21 | +22.1% |
| 0.3 | 153 | 226 | 273 | +20.8% | 2.28 | 1.78 | 1.77 | −0.6% |
| 3.0 | 192 | 270 | 281 | +4.1% | 2.15 | 2.16 | 1.19 | −44.9% |
| 30 | 184 | 294 | 268 | −8.8% | 1.85 | 2.18 | 1.07 | −50.9% |
| | Total cholesterol (mmol/l) | | | | HDL-cholesterol (mmol/l) | | | |
| Vehicle | 4.33 | 4.20 | 3.83 | −8.8% | 2.58 | 2.93 | 2.82 | −3.8% |
| 0.3 | 4.52 | 4.05 | 3.82 | −5.7% | 2.43 | 3.00 | 2.85 | −5.0% |
| 3.0 | 4.62 | 3.95 | 4.39 | −11.1% | 2.47 | 2.88 | 3.41 | +18.4% |
| 3.98 | 3.98 | 4.27 | 4.53 | +6.1% | 2.45 | 3.07 | 3.39 | +10.4% |

In the groups with 0.3 mg/kg/day there is no significant change when compared to the vehicle control.

In the group with 3 mg/kg/day there is no significant change of glucose levels by compounds 1 and 2, whereas compound 15 markedly lowers glucose levels. Triglyceride levels are only markedly decreasing after treatment with compound 15, but are hardly influenced by compounds 1 and 2. Total cholesterol levels are not markedly changed by any of the 3 compounds. HDL-cholesterol levels are markedly raised by compound 15, but not influenced by compounds 1 and 2.

In the groups treated with a dose of 30 mg/kg/day the results are clear-cut. Glucose levels decrease markedly in the case of administration of compounds 1 and 15, but are only hardly influenced by compound 2. Triglyceride levels are decreasing significantly with compound 1 and very strongly with compound 15, whereas compound 2 even raises triglyceride levels. Total cholesterol levels are slightly lower with compound 2 and slightly higher with compounds 1 and 15. HDL-cholesterol is markedly increased by compounds 1 and 15 and decreased by the treatment with compound 2.

Conclusion: Compounds 1 and 15 have a very favourable influence on glucose and lipid metabolism whereas compound 2 only stabilizes glucose metabolism, but deteriorizes lipid metabolism by increasing triglycerides and decreasing HDL-cholesterol.

This can (without it being desired to be bound to this explanation throughout this disclosure) be explained in the following way: Compound 1 and 15 are pure RXR agonists or antagonists, whereas compound 2 has, besides RXR, also RAR agonistic activity, which latter is known to be responsible for the deterioration of the lipid metabolism.

In Table 6 the overall evaluation is presented, comparing the groups treated with 30 mg/kg/day with the vehicle group.

TABLE 6
Investigations with db/db mice
Treatment with Compounds 1, 2 and 15
For overall evaluation of efficacy the extent of the effect on blood
levels of glucose, triglycerides, total cholesterol and HDL-cholesterol
was determined by including the data of the vehicle groups and those
of the groups treated with 30 mg/kg/day.

| | Blood levels of: | | | |
|---|---|---|---|---|
| | | | Total | HDL- |
| | Glucose | Triglycerides | cholesterol | cholesterol |
| Compound 1 | −62.5% | −21.5% | −16.6% | +18.4% |
| Pure RXR | | | | |
| agonist | | | | |
| Compound 2 | +5.6% | +18.3% | −13.0% | −17.8% |
| Partial RXR | | | | |
| agonist | | | | |
| Compound 15 | −58.0% | −73.0% | +14.9% | +14.2% |
| Pure RXR | | | | |
| antagonist | | | | |

Table 6 shows efficacy calculated from the whole extent of effect. Regarding a favourable effect on glucose metabolism, compounds 1 and 15 are markedly superior to compound 2. Regarding the effect on lipids, compounds 1 and 15 have a favourable effect by decreasing triglycerides and increasing HDL-cholesterol, whereas compound 2 increases triglycerides and decreases HDL-cholesterol. These results explain the clinical failure of compound 2 for treatment of metabolic diseases, since high triglycerides and low HDL-cholesterol favour the development of atherosclerosis. Such compounds are prohibitive for treatment of diseases with a high risk for atherosclerosis.

Based on these results, it is concluded that the mentioned compounds, in particular those mentioned above as preferred for USE, more particularly compounds 1 and 15 and their analogs, with RXR agonistic or antagonistic activity are capable to influence favourably the therapeutic indications presented in this invention. These retinoids cover the same spectrum of metabolic and cardiovascular diseases as do the ligands to PPARs, α, β/δ and γ. Therefore, the combination of any of these retinoids with any of the PPAR ligands possess an either additive or super-additive, synergistic effect in prevention and therapy of the above mentioned diseases 1 to 6, diabetes type II, obesity, dyslipidemia, hypertension, atherosclerosis and polyneuropathy.

### Determination of Compounds for Use in Treatment of Metabolic Diseases and Cardiovascular Diseases

Tailor made compounds or combination of compounds for treatment of metabolic diseases and cardiovascular diseases, mainly diabetes type II, obesity, dyslipidemia and atherosclerosis, are also determined on the basis of molecular biology experiments.

Up to the present time the clinical treatment with ligands to the nuclear hormone receptors PPARs has led to remarkable but still limited therapeutic results in the above mentioned diseases. However, in spite of some success in clinical use with favourably influencing either glucose metabolism/insulin resistance, dyslipidemia or cardiovascular parameters, they often also cause undesired side effects, for instance liver toxicity or weight gain.

It is a purpose of this invention to present compounds or combinations of compounds, possessing a maximum of desired favourable effects associated with a minimum of undesired toxic side effects and adverse events. It can probably not be expected that any of the compounds given as single agent or any of the possible combination of compounds will be able to treat successfully the whole spectrum of therapeutic indications without causing any of the mentioned side effects. Nevertheless, in the present invention the use of the ligands to RXR, RXR agonists and RXR antagonists, compounds 1, and 4 to 24, especially the compounds mentioned as preferred above (that is, with the exception of the compounds preferably excepted) alone or their combination with ligands to PPARs α, β/δ and γ, is described, for treatment of diseases falling under the metabolic syndrome, especially of diabetes type II, obesity, dyslipidemia and atherosclerosis. It may be anticipated that every single RXR ligand or particularly each sort of combination of RXR ligands and PPAR ligands will optimally influence only one or a part of all the mentioned therapeutic indications without inducing side effects, but complete efficiency cannot be excluded here.

It is important to mention that two compounds, compound 2 and 3 had originally been chosen by other authors on the basis of animal experiments with db/db mice for clinical trials (Mukherjee et al. Arterioscler. Thromb. Vasc. Biol. 1998, 18:272-276). Unfortunately, in humans, despite of influencing favourably the glucose metabolism, the compounds led to a marked triglyceridemia which is, in fact, prohibitive for treatment of diabetes, because of its high risk for developing atherosclerosis.

The other RXR agonists and RXR antagonists, mentioned in the list of Tables 1 and 2, in particular compounds 1 and 4 to 14, compounds 15 to 20 and compounds 21 to 24, especially the compounds mentioned as preferred above, are good candidates particularly in combination with PPAR ligands for a successful treatment of metabolic and cardiovascular diseases.

For choosing the most favourable compounds or particularly the most favourable combination the following molecular biological investigations are undertaken, being predictive for the various therapeutic areas of metabolic and cardiovascular diseases.

The nuclear hormone receptors are ligand-dependent activated transcription factors regulating critical pathways essential in physiology and pathology of mammals. The RXRs play a central role in many functions through their ability to act as obligatory heterodimer partners for many members of the nuclear receptor family including in particular the PPARs α, β/δ and γ. Among the heterodimer and homodimer formations, various possibilities exist by choosing ligands with either agonistic or antagonistic activity.

In the following example 5, functional transactivation assays were used in particular the assay characterizing hits from the primary ligand binding screen of the GAL 4-LBD (ligand binding domain) transactivation assay.

### EXAMPLE 5

### Transactivation Assays

### Method

Nuclear hormone receptor (NHR) ligand dependent transcriptional transactivation assay.

This secondary in vitro functional assay to characterize hits from the primary ligand binding screens is a GAL4-LBD transactivation assay. The Gal4-LBD protein consists of an in frame fusion of the transcription factor galactose 4 DNA binding domain from S.cerevisiae with the ligand binding domain (LBD) of a particular NHR. Briefly, a plasmid construct expressing the recombinant chimeric receptor is co-transfected into mammalian cells (BHK21, CV1, etc) with a luciferase reporter plasmid containing several copies in tandem of the GAL4 binding sites (promoter recognition elements) upstream from the minimal promoter driving the luciferase gene. The transfected cells are treated with compounds for 12-24 hours, lysates are produced and assayed for luciferase activity and normalized to internal standards such as GH, growth hormone or SEAP, secreted alkaline phosphatase. Ligand binding stabilizes the LBD conformation that recruits endogenous co-factors that mediate eventually the increased transcriptional activity of the luciferase reporter gene. By comparing the activation profile of a reference ligand or a natural ligand, the EC50 values for agonistic activity of a particular ligand can be measured. The extent of activation that can be measured is indicative of a partial, respectively full agonistic activity of a ligand.

Heterodimer reporter assays are potentially more important selection criteria for synthetic agonistic NHR ligands as they embody a more natural situation. Basically plasmids expressing the recombinant full length NHR are co-transfected with plasmids expressing the full length RXR heteropartner receptor and a reporter plasmid containing several copies of the natural responsive elements for the respective NHR driving the luciferase reporter gene. (For a reference see: Mukherjee R, Jow L. Noonan and Mc Donnell D P. Human and rat peroxisome proliferator activated receptors (PPARs) demonstrate similar tissue distribution but different responsiveness to PPAR activation. J Steroid Biochem Mol Biol 1994; 51: 157-166.

TABLE 7
RXR Agonists
with RXRα/RXRα Homodimer Transactivation and
with PPARγ/RXRα Heterodimer Transactivation
(Type Compound 1)

| RXRα Agonist | Comp 1 | Comp 2 | Comp 3 | Comp 4 | Comp 5 | Comp 6 | Comp 7 |
|---|---|---|---|---|---|---|---|
| RXRα | 110 | 110 | 30 | 84 | 150 | 330 | 780 |
| Binding Affinity | | | | | | | |
| IC50 (nmol) | | | | | | | |
| RXRα Homodimer | 1.4 | 5 | <1 | 3.5 | 0.8 | 19 | 6 |
| Activation Assay | | | | | | | |
| EC50 (nmol) | | | | | | | |
| PPARγ/RXRα Heterodimer | 70 | 6 | 5 | | 1.5 | | |
| Activation Assay | | | | | | | |
| EC50 (nmol) | | | | | | | |
| RXRα Agonist | Comp 8 | Comp 9 | Comp 10 | Comp 11 | Comp 12 | Comp 13 | Comp 14 |
| RXRα | 100 | 230 | 110 | 65 | 80 | 184 | 110 |
| Binding Affinity | | | | | | | |
| IC50 (nmol) | | | | | | | |
| RXRα Homodimer | 2 | 2.5 | 2.6 | 2 | 9 | 1.5 | 6 |
| Activation Assay | | | | | | | |
| EC50 (nmol) | | | | | | | |
| PPARγ/RXRα Heterodimer | 14 | 38 | 6 | 10 | 7.7 | 23 | 7 |
| Activation Assay | | | | | | | |
| EC50 (nmol) | | | | | | | |

RXRα Antagonists
with no RXRα/RXRα Homodimer Transactivation and
with no PPARγ/RXRα Heterodimer Transactivation
(Type Compound 15)

| RXRα Antagonist | Comp 15 | Comp 16 | Comp 17 | Comp 18 | Comp 19 | Comp 20 |
|---|---|---|---|---|---|---|
| RXRα | 30 | 65 | 49 | 95 | 9 | 52 |
| Binding Affinity | | | | | | |
| IC50 (nmol) | | | | | | |
| RXRα Homodimer | >10,000 | >10,000 | >10,000 | >10,000 | >10,000 | >10,000 |
| Activation Assay | | | | | | |
| EC50 (nmol) | | | | | | |
| PPARγ/RXRα Heterodimer | >10,000 | >10,000 | >10,000 | >10,000 | >10,000 | >10,000 |
| Activation Assay | | | | | | |
| EC50 (nmol) | | | | | | |

RXRα Antagonists
with no RXRα/RXRα Homodimer Transactivation
but with PPARγ/RXRα Heterodimer Transactivation
(Type Compound 21)

| RXRα Antagonist | Comp 21 | Comp 22 | Comp 23 | Comp 24 |
|---|---|---|---|---|
| RXRα | 24 | 300 | 30 | 680 |
| Binding Affinity | | | | |
| IC50 (nmol) | | | | |
| RXRα Homodimer | >10,000 | >10,000 | >10,000 | >10,000 |
| Activation Assay Homodimer | | | | |
| EC50 (nmol) | | | | |
| PPARγ/RXRα Heterodimer | 1.25 | >30 | 6 | 3.5 |
| Activation Assay | | | | |
| EC50 (nmol) | | | | |

A. The RXR agonists transactivate the RXR homodimer as well as the PPARγ/RXR heterodimer. These compounds are called RXR agonists of type compound 1. The group includes, besides compound 1, the compounds 4 to 14. All of them transactivate the RXR homodimer very markedly with EC50 varying between 0.8 and 19 nmol. They transactivate also the PPARγ/RXR heterodimer with EC50 varying between 1.5 and 70 nmol.

These results provide evidence that the RXR agonists, compounds 1, and 4 to 14 have the potential to act in a similar way as the ligands to PPAR γ. Since PPARγ functions as a regulator of glucose metabolism, the combination of RXR agonists and PPARγ ligands can be expected to have an additive or even a synergistic beneficial effect on glucose metabolism. As a consequence such a combination is useful for treatment of diabetes type II. This combination therapy is not limited to the treatment of diabetes, since PPARγ ligands and also PPARα ligands have an additional beneficial effect on lipid metabolism. The therapy is therefore useful particularly for treatment of diabetes type II and dyslipidemia.

B. The RXR antagonists, investigated with molecular biology experiments, can be classified into two classes having different properties.
B.1. The RXR antagonists, compounds 15 to 20, called type compound 15 do neither transactivate RXR homodimer, nor do they transactivate the PPARγ/RXR heterodimer. They are pure RXR antagonists. This group of compounds, in particular compound 15, has been proven to exert marked anti-inflammatory effects when administered topically or orally to mammals with inflammatory manifestations in various tissues and organs, as described in examples 6 to 9 and 20 to 24. Beside the known predisposing factors for development of atherosclerosis such as diabetes, obesity, dyslipidemia and hypertension, inflammatory processes contribute to development of atherosclerosis (Barish GD et al. Trends in Endocrinology and Metabolism 2004; 15:158-165).
Inflammatory processes play a dominant role in pathogenesis of atherosclerosis. Therefore, this class of compounds is particularly useful for prevention and therapy of atherosclerosis as single agents or even better in combination with ligands to PPARα and γ, but also to PPAR β/δ which latter is also markedly involved in controlling inflammatory responses. In regulation of inflammatory processes all PPAR isotypes α, β/δ and γ are anti-inflammatory transcription factors and act via macrophages, dendritic cells, B cells, T cells and cytokines, contributing to inhibition of many inflammation-inducing stimuli (Genolet R. et al. Current Drug Targets—Inflammation and Allergy 2004; 3: 365-375).
Since atherosclerosis is partly caused by inflammatory processes, it can be concluded that anti-inflammatory agents such as the RXR antagonist compound 15 or analogs as well as ligands to any of the PPARα, β/δ or γ are useful in the prevention and therapy of atherosclerosis.
Atherosclerotic lesions are not only considered as deposits of excess lipids in the vascular wall. They are sites of chronic inflammation. The invasion of monocytes into the arterial wall and their subsequent differentiation into cholesterol-laden macrophages, known as foam cells is a central feature of atherosclerotic disease.
Excess cholesterol is eliminated predominantly by reverse cholesterol transport. In this process free cholesterol is removed via efflux to extracellular acceptors or apolipoproteins and converted to HDL-cholesterol. The switch from the “bad” low density lipoproteins (LDL) to the “good” high density lipoprotein (HDL) is a particular advantage of the RXR antagonists.
In addition to trafficking cholesterol, macrophages secrete inflammatory cytokines and matrix metalloproteinases (MMPs). These mediators crosstalk with T-cells and vascular cells to modify the extracellular space and induce further inflammation and formation of complex highly cellular atherosclerotic lesions.
The RXR antagonists in particular compounds 15 and analogs have the rather unique property of favorably influencing the lipid metabolism and possessing in addition, marked anti-inflammatory properties. They are, therefore, predestinated for the successful treatment of metabolic and cardiovascular diseases and in particular for prevention and treatment of atherosclerosis.
B.2. The RXR antagonists, compounds 21 to 24, called type compound 21 do not transactivate RXR homodimer, but transactivate the PPAR γ/RXR heterodimer. They are mixed or partial RXR agonists/antagonists. They transactivate the PPARγ/RXR heterodimer with EC50 varying between 1.95 and about 30 nmol.
These results provide evidence that the RXR antagonists, type compound 21 have the potential to act in a similar way as the ligands to PPARγ. Since PPARγ functions as a regulator of glucose metabolism, these RXR antagonists have a beneficial effect when administered as single agents or even better in combination with a PPARγ ligand for achieving an additive or synergistic effect. The therapy with RXR antagonists type compound 21, is not limited to the treatment of diabetes type II, since PPARγ ligands have, not only a beneficial effect on glucose metabolism, but as well on lipid metabolism. The therapy with these RXR antagonists as single agents or in combination with PPAR γ ligands are therefore useful in therapy of diabetes as well as for therapy of lipid disorders. Also the combination of this class of RXR antagonists with ligands of PPARs α or β/δ can be useful in therapy of metabolic and cardiovascular diseases.

The difference in results of molecular binding experiments with the two groups of RXR antagonists (see B.1. above, compounds 15 to 20 and B.2., compounds 21 to 24) points out that these two groups of RXR antagonists have a different spectrum of therapeutic indications within the frame of diseases of the metabolic syndrome and of cardiovascular diseases.

### EXAMPLES 6 and 7

### Acute and Semichronic Inflammation

Inflammation is induced by topical (epicutaneous) application of retinoid receptor agonists e.g. retinoic acids all-trans retinoic acid (AtRA) or 9-cis retinoic acid (9-cis RA); or especially (this forming a case with a totally different etiology for the inflammation based on protein kinase C) by the topical application of the phorbolester 12-O-tetradecanoylphorbol-13-acetate (TPA).

### Methods

Nude mice of the C57BL/6 strain are used. Inflammation is induced on mouse ears with either AtRA, 9-cis RA or TPA by topical application. Inflammation is measured objectively by determination of the activity of myeloperoxidase (MPO) being directly correlated to the infiltration of polymorphonuclear white blood cells and by the determination of mRNA expression of c-jun, a protein implicated in the AP-1 transduction pathway according to known methods, see e.g. P. L. Stanley et al., Skin Pharmacol. 4, 262-271 (1991) (MPO assay), N. Basset-Sequin et al., J. Invest. Dermatol. 94, 418-422 (1990), F. J. Rauscher et al., Cell 52, 471-480 (1982), P. Sassone-Corn et al., Nature 326, 507-510 (1987), and M. Pfahl, Endocr. Rev. 14, 651-658, 1993, which are incorporated by reference regarding the experimental method.

In the “acute inflammation” test, mice are treated topically (epicutaneously), orally or intraperitoneally, daily for 4 days. In the “semi chronic inflammation” test, mice are treated topically according to the schedules given below. For each experiment, a group of at least 4 mice of both sexes are used in the defined condition, regarding placebo, vehicle control, compound, topical formulation, oral formulation, dosage and concentration. The topical vehicle consists of ethanol/PEG 400/water (3:1:1).

### Results:

### EXAMPLE 6

### Acute Inflammation

The anti-inflammatory effect of topical RXR antagonist compound 15 is tested by determination of myeloperoxidase activity in % of vehicle treated controls. For induction of inflammation 9-cis RA or TPA are applied topically to the skin, daily for 4 days. The RXR antagonist compound 15 (see Table 1) is administered topically one hour after the application of the inflammation inducing agent. The mice are sacrified 24 hours after the last treatment. The results are presented in Table 8:

TABLE 8

| | MPO activity |
|---|---|
| Ears C57BL/6 | (% vehicle treated mice) |
| 9-cis RA 0.05% (4 d) | 647 ± 142 |
| 9-cis RA 0.05% + compound 15 0.05% (4 d) | 236 ± 92 |
| TPA 0.005% (4 d) | 376 ± 72 |
| TPA 0.005% + compound 15 0.05% (4 d) | 163 ± 25 |
| TPA 0.005% + compound 15 2.5% (4 d) | 142 ± 18 |

As can be seen from Table 8, topical administration of compound 15 significantly decreases the MPO activity induced by prior aplication of topical 9-cis RA or topical TPA.

### EXAMPLE 7

### Semi Chronic Inflammation

The anti-inflammatory effect of topical RXR antagonists is tested by determination of myeloperoxidase activity in % of vehicle controls. The effect of the RXR antagonist compound 15 is also compared with the well known anti-inflammatory effect of the two corticosteroids, clobetasol dipropionate and betamethasone propionate. For induction of inflammation, AtRA and TPA are applied topically to the skin and the administration of the test compounds, the RXR antagonist compound 15 and the two corticosteroids are given in the following order, according to the schedule, described in: Skin Pharmacol 1991; 4 (4): 262-271, Stanley P L. et.al.: RA or TPA is administered on days 0, 2, 4, 7 and 9, compound 15 or corticosteroids are administered twice daily on day 7, day 8 and day 9 and once on day 10 in the morning. The mice are sacrificed on day 10 in the afternoon. The results are presented in Table 9a.

TABLE 9a

| | MPO activity |
|---|---|
| Ears C57BL/6 | (% vehicle treated mice) |
| At RA 0.05% (11 d) | 1371 ± 345 |
| At RA 0.05% + compound 15 0.05% (11 d) | 184 ± 72 |
| TPA 0.005% (11 d) | 572 ± 61 |
| TPA 0.005% + compound 15 0.05% (11 d) | 345 ± 81 |
| TPA 0.005% + Clobetasol dipropionate | 239 ± 43 |
| 0.05% (11 d) | |
| TPA 0.005% + Betamethasone propionate | 257 ± 38 |
| 0.05% (11 d) | |

As can be seen from Table 9a, topical administration of compound 15 significantly decreases the MPO activity induced by prior application of topical 9-cis RA or topical TPA. The two corticosteroids have a rather similar effect on the TPA-induced skin inflammation. This shows that the RXR antagonists do not only compensate the adverse effects of retinoic acid but are more generally applicable to treat inflammations.

In the same experiment, the expression of c-Jun mRNA is determined by northern blot and expressed in percent of the vehicle controls. The results are presented in Table 9b:

TABLE 9b

| | c-Jun mRNA expression |
|---|---|
| Ears C57BL/6 | (% of vehicle treated mice) |
| Vehicle | 100 |
| TPA 0.005% | 163 |
| TPA 0.005% + Betamethasone 0.05% | 24 |
| TPA 0.005% + Clobetasol 0.05% | 43 |
| TPA 0.005% + compound 15 0.05% | 88 |

As can be seen from Table 9b, topical administration of compound 15 inhibits the c-Jun mRNA expression. The corticosteroids also decrease the expression of c-Jun mRNA. In the same concentration of 0.05% they have a stronger inhibitory effect than compound 15.

However, it has to be taken in consideration that compound 15 can be applied epicutaneously in much higher concentrations than the corticosteroids without inducing cutaneous adverse events.

TPA-induced inflammation is known to be transduced by AP-1 pathway. A partly common mechanism of action of RXR antagonists and corticosteroids may be possible, based on the repression of c-Jun expression and the correlated inhibition of myeloperoxidase.

### EXAMPLE 8

### Effect on Normal, Non-Inflamed Skin

### Methods

The effect of the RXR antagonist compound 15 on normal skin of mice is investigated. The ears of C57BL/6 mice are treated epicutaneously (topically) for 4 consecutive days with compound 15 in a concentration of 0.05% and 2.5% in acetone/ethanol (1:1, v/v). Comparison with vehicle control. Skin reactions, in particular inflammation, erythema, desquamation, and edema, are observed daily. Myeloperoxidase (MPO) activity is determined. The activity of MPO is considered the most sensitive criterium for the assessment of inflammation of the skin (see above, Examples 6 and 7).

### Results

Compound 15 does not induce clinical signs or symptoms of a skin inflammation on normal skin of mice. At a concentration of 0.05% of compound 15, there is no significant change of MPO activity compared to the vehicle control. However, at a concentration of 2.5% of compound 15, MPO activity is significantly diminished to 57% of that of the vehicle control. The conclusion may be drawn, that compound 15 in higher, but still well tolerated concentrations even decreases the basal activity of MPO in normal skin. This provides evidence to indicate a preventive effect of compound 15 towards inflammatory agents or in patients with inflammatory skin diseases in case of exposition to inflammation-inducing agents.

### Examples for Inflammatory Diseases of Bones and Joints:

### EXAMPLE 9

### Effect of RXR Antagonists on Degradation/Destruction of Human Cartilage Induced by Synovial Fibroblasts taken from Patients with Rheumatoid Arthritis. Ex Vivo, in vitro Model System for Rheumatoid Arthritis (RA) and Osteoarthritis (OA).

### Methods:

The effect of compound 15 (RXR antagonist) on the activity of synovial fibroblasts, dependent on their state of activation, i.e. modified by a concomitant stimulation by the inflammatory cytokine Interleukin-1β (IL-1β), is determined. Furthermore, it is determined whether this is accompanied by a modulation in the accumulation of the mRNA encoding catabolic enzyme matrix metalloproteinase-1 (MMP-1), responsible for degradation of human cartilage and consequently joint destruction in man. Adherent synovial fluid cells taken from a patient with RA are used after 5 passages in an in vitro assay for cartilage destruction. The cells incubated in flasks coated with 0.1% (0.1 g/100 ml) human cartilage powder are fixed using Matrigel® (BD Biosciences, Becton, Dickinson & Co., Boston, Mass., USA). The release of sulphated glycosaminoglycan (sGAG) into the culture medium is monitored by a commercial colorimetric test according to a method described by S. Björnsson, see Anal. Biochem. 256, 229-237 (1998) using an alcian blue dot plot analysis, and the accumulation of mRNA encoding MMP-1 is quantified by real time PCR (TaqMan® (Roche Diagnostics, Basle, Switzerland)).

The retinoid agonists all-trans retinoic acid and 9-cis retinoic acid, both physiological metabolites of vitamin A, as well as the RXR antagonist compound 15, diluted first in ethanol, and then diluted with vehicle or medium to the desired dose/concentration are tested in a time course (0-35 days for the in vitro assay, 0-48 hours for MMP-1 mRNA, see tables 12, 13 and 15) and dose-dependent (10⁻⁷ to 10⁻⁹ M, see tables 10, 11 and 14). This is conducted in the presence or absence of IL-1β (100 pg/ml).

### Results

In the absence of IL-1β, the retinoid pan agonist 9-cis RA increases cartilage destruction in vitro in a dose-dependent manner (maximal between 10⁻⁷ M and 10⁻⁸ M), whereas the RXR antagonist compound 15, in contrast, has no effect on the basal activity of synovial fibroblast (Table 10).

TABLE 10
In vitro cartilage degradation. Dose dependency.
Effect of 9-cis retinoic acid (9-cis RA) versus compound 15
(RXR antagonist) in absence of IL-1β. Release of sGAG in
μg/ml/14 days.

sGAG in μg/ml

| Dose/Concentration | 9-cis RA | compound 15 |
|---|---|---|
| Vehicle control | 48 | 48 |
| 10⁻⁹ M | 64 | 46 |
| 10⁻⁸ M | 107 | 57 |
| 10⁻⁷ M | 189 | 39 |

However in the presence of IL-1β, quite surprisingly, the RXR antagonist compound 15 markedly inhibits the IL-1β dependent cartilage destruction, evidenced by a decrease in sGAG (Table 11).

TABLE 11
In vitro cartilage degradation. Dose dependency.
Effect of 9-cis RA versus compound 15 (RXR antagonist) in
presence of 100 pg/ml IL-1β. Release of sGAG in μg/ml/14 days.

sGAG in μg/ml

| Dose/Concentration | 9-cis RA | compound 15 |
|---|---|---|
| Vehicle control | 173 | 173 |
| 10⁻⁹ M | 204 | 144 |
| 10⁻⁸ M | 189 | 89 |
| 10⁻⁷ M | 221 | 41 |

The time course confirms that the retinoid agonist 9-cis RA markedly increases cartilage destruction in vitro, whereas with the retinoid antagonist compound A this is not the case. This effect is observed both in the presence and absence of IL-1β (Tables 12 and 13):

TABLE 12
In vitro cartilage degradation. Time dependency.
Effect of 9-cis RA versus compound 15 (RXR antagonist) in
absence of IL-1β.
Release of sGAG in μg/ml/14 days.

| | Cumulative μg/ml sGAG/14 days | | |
|---|---|---|---|
| | | 9-cis RA | compound 15 |
| Days | Control | 10⁻⁸ M | 10⁻⁸ M |
| 7 | 23 | 44 | 44 |
| 14 | 48 | 107 | 57 |
| 21 | 84 | 206 | 57 |
| 28 | 112 | 253 | 39 |
| 35 | 117 | 292 | 34 |

TABLE 13
In vitro cartilage degradation. Time dependency.
Effect of 9-cis RA versus compound 15 (RXR antagonist) in
presence of 100 pg/ml IL-1β. Release of sGAG in μg/ml/14 days.

| | Cumulative μg/ml sGAG/14 days | | |
|---|---|---|---|
| | | 9-cis RA | compound 15 |
| Days | Control | 10⁻⁸ M | 10⁻⁸ M |
| 7 | 86 | 68 | 67 |
| 14 | 173 | 189 | 89 |
| 21 | 212 | 330 | 89 |
| 28 | 249 | 407 | 173 |
| 35 | 271 | 441 | 56 |

Finally, the cartilage destruction in vitro correlates well with the accumulation of MMP-1 mRNA in synovial fibroblasts incubated for 12 hours. (Tables 14, 15):

TABLE 14
Matrix metalloproteinase-1 (MMP-1) production. Dose dependency.
Effect of 9-cis RA versus compound 15 (RXR antagonist) MMP-1
mRNA (real time PCR, fold increase of baseline value, after 24 hours)
in relative units.

MMP-1 mRNA in
relative units

| Dose/Concentration | 9-cis RA | compound 15 |
|---|---|---|
| Vehicle control | 1 | 1 |
| 10⁻⁹ M | 1.54 | 1.05 |
| 10⁻⁸ M | 3.39 | 1.12 |
| 10⁻⁷ M | 2.60 | 0.68 |

TABLE 15
Matrix metalloproteinase-1 (MMP-1) production. Time dependency.
Effect of 9-cis RA versus compound 15 (RXR antagonist) MMP-1
mRNA (real time PCR, fold increase of baseline value, after 0 to
24 hours) in relative units.

| | MMP-1 mRNA in relative units | | |
|---|---|---|---|
| | | | compound 15 |
| Hours | Control | 9-cis RA 10⁻⁸ M | 10⁻⁸ M |
| 0 | 1 | 1 | 1 |
| 2 | | 0.98 | 1.13 |
| 6 | | 1.31 | 0.81 |
| 12 | 1.13 | 3.39 | 1.12 |
| 24 | | 3.79 | 0.87 |
| 48 | 1.09 | 1.47 | 0.83 |

### Conclusion

RXR antagonists inhibit cartilage destruction in a pharmacological model system for destruction of joints in rheumatoid arthritis and osteoarthritis.

### Examples of Pharmaceutical Formulations for Treating Inflammatory Diseases by Topical or Oral Administration of RXR Antagonists:

The following formulations useful for USE in the present invention are prepared according to the tables presented and using standard procedures or the procedures specifically mentioned in Examples 10 to 13 for oral and in Examples 14 to 18 for topical administration, where “Active compound” stands for any one of the compounds mentioned in Table 1, especially compound 15 or more especially compound 1:

### EXAMPLE 10

### Fill Mass for Soft Gelatin Capsules and Capsules Filled with Said Fill Mass

A fill mass for soft gelatine capsules is prepared using the following components:

TABLE 16a
a) Fill mass for soft gelatin capsules

| | | |
|---|---|---|
| Active compound | 10-200 | g |
| Oil* | 1-3 | parts |
| Wax mixture** | 1-5 | parts |
| Fill volume | 1-6 | minims |

*natural vegetable oils, e.g. soy oil, peanut oil, and artificial glycerides
**composition of natural and artificial waxes or partially hydrogenated fats

This fill mass is then used to produce soft gelatine capsules with the following content:

TABLE 16b
b) Soft gelatine capsules containing 20-100 mg active substance
20 mg soft gelatine capsule

| Ingredients | mg/capsule | |
|---|---|---|
| Active compound | 20.000 | |
| dl-α-Tocopherol | 0.028 | |
| Hydrogenated Castor Oil | 4.200 | |
| Caprylic/Capric/Stearic Triglyceride | 56.000 | |
| (Synthetic Triglyceride) | | |
| Triglyceride, Medium Chain | 199.772 | |
| Total | 280.000 | mg |

### EXAMPLE 11

### Hard Gelatin Capsules

Hard gelatine capsules are prepared as follows:

TABLE 17
Hard gelatine capsules containing 20-100 mg active substance
20 mg hard gelatine capsule

| Ingredients | mg/capsule | |
|---|---|---|
| Active compound | 20.0 | mg |
| Gelatine Bloom 30 | 70.0 | mg |
| Maltodextrin MD 05 | 108.0 | mg |
| dl-α-Tocopherol | 2.0 | mg |
| Sodium ascorbate | 10.0 | mg |
| Microcrystalline cellulose | 48.0 | mg |
| Magnesium stearate | 2.0 | mg |
| (weight capsule content) | 260.0 | mg |

Procedure:
The active substance is wet milled in a solution of gelatine, maltodextrin, dl-α-Tocopherol and sodium ascorbate. The wet milled suspension is spray-dried.
The spray-dried powder is mixed with microcrystalline cellulose and magnesium stearate. 260 mg each of this mixture are filled into hard gelatine capsules of suitable size and color.

### EXAMPLE 12

### Tablets

Tablets are prepared as follows:

TABLE 18
Tablets containing 20-50 mg active substance
20 mg tablet

| | mg/tablet | |
|---|---|---|
| Tablet kernel | | |
| Active compound | 20.0 | mg |
| Anhydrous lactose | 130.5 | mg |
| Microcrystalline Cellulose | 80.0 | mg |
| dl-α-Tocopherol | 2.0 | mg |
| Sodium ascorbate | 10.0 | mg |
| Polyvinylpyrrolidone K30 | 5.0 | mg |
| Magnesium stearate | 2.5 | mg |
| (Kernel weight) | 250.0 | mg |
| Film coat | | |
| Hydroxypropyl methylcellulose | 3.5 | mg |
| Polyethylenglycol 6000 | 0.8 | mg |
| Talc | 1.3 | mg |
| Irone oxide, yellow | 0.8 | mg |
| Titanium dioxide | 0.8 | mg |
| (weight of film) | 7.4 | mg |

Procedure:
The compound is mixed with anhydrous lactose and microcrystalline cellulose. The mixture is granulated in water with a solution/dispersion of polyvinylpyrrolidone, dl-α-Tocopherol and sodium ascorbate. The granular material is mixed with magnesium stearate and afterwards pressed as kernels with 250 mg weight. The kernels are film coated with a solution/suspension of above-mentioned compositions.

### EXAMPLE 13

### Sachets

Sachets are prepared with the following ingredients:

TABLE 19
Sachets containing 200-500 mg active substance
200 mg sachet

| Ingredients | mg/sachet | |
|---|---|---|
| Active compound | 200.0 | mg |
| Lactose, fine powder | 990.0 | mg |
| Microcrystalline Cellulose | 1250.0 | mg |
| Sodium Carboxymethyl cellulose | 14.0 | mg |
| dl-α-Tocopherol | 5.0 | mg |
| Sodium ascorbate | 20.0 | mg |
| Polyvinylpyrrolidone K30 | 10.0 | mg |
| Magnesium stearate | 10.0 | mg |

### EXAMPLE 14

### Lotion, Solution or Suspension

A lotion, solution or suspension is prepared with the following composition:

TABLE 20
Lotion, solution or suspension

| | | | Preferred | |
|---|---|---|---|---|
| Active compound | 0.3-2.0 | g | | |
| Propylene Glycol | 5.00-20.00 | g | 10.00 | g |
| PEG-Glyceryl Cocoate* | 0.00-20.00 | g | 10.00 | g |
| dl-α-Tocopherol | 0.001-0.50 | g | 0.02 | g |
| Ascorbyl Palmitate | 0.01-0.20 | g | 0.10 | g |
| Propyl Gallate | 0.001-0.02 | g | 0.002 | g |
| Citric acid, anhydr.** | 0.00-0.20 | g | 0.01 | g |
| Isopropanol*** | 40.00-90.00 | g | 50.00 | g |
| Water, dem. ad | 100.00 | g | 100.00 | g resp. ml |

*or other tensides
**or other complexing agents e.g. EDTA
***or other alcohols e.g. ethanol

### EXAMPLE 15

### Gel

A gel is prepared with the following composition:

TABLE 21
Gel

| | | | preferred | |
|---|---|---|---|---|
| Active compound | 0.3-2.0 | g | | |
| Propylene Glycol | 5.00-20.00 | g | 10.00 | g |
| PEG-Glyceryl Cocoate* | 0.00-20.00 | g | 10.00 | g |
| dl-α-Tocopherol | 0.001-0.50 | g | 0.02 | g |
| Ascorbyl Palmitate | 0.01-0.20 | g | 0.10 | g |
| Propyl Gallate | 0.001-0.02 | g | 0.002 | g |
| Citric acid, anhydr.** | 0.00-0.20 | g | 0.01 | g |
| Isopropanol*** | 40.00-90.00 | g | 50.00 | g |
| HPMC**** | 0.50-5.00 | g | 3.00 | g |
| Preservative***** | q.s. | | q.s. | |
| Water, dem. ad | 100.00 | g | 100.00 | g resp. ml |

*or other tensides
**or other complexing agents e.g. EDTA
***or other alcohols e.g. ethanol
****Hydroxypropyl methylcellulose or other polymers e.g. neutralised Carbomer, Methyl cellulose, sodium carboxymethylcellulose
*****Preservatives e.g. paraben esters (methyl, ethyl, propyl, butyl), sorbic acid and/or benzoic acid.

### EXAMPLE 16

### Cream

A cream is manufactured with the following composition:

TABLE 22
Cream

| | | | preferred | |
|---|---|---|---|---|
| Active compound | 0.3-2.0 | g | | |
| Glycerol | 0.00-10.00 | g | 5.00 | g |
| Na₂•EDTA | 0.001-0.50 | g | 0.03 | g |
| Glycerides* | 5.00-20.00 | g | 10.00 | g |
| Cetyl Alcohol | 0.50-5.00 | g | 1.00 | g |
| Stearyl Alcohol | 0.50-5.00 | g | 1.00 | g |
| Glycerol mono Stearate | 1.00-8.00 | g | 4.00 | g |
| Ceteareth** | 0.50-5.00 | g | 2.00 | g |
| dl-α-Tocopherol | 0.001-0.50 | g | 0.02 | g |
| Preservative*** | q.s. | | q.s. | |
| Water, dem. ad | 100.00 | g | 100.00 | g res. ml |

*e.g. caprylic/capric/triglyceride, caprylic/capric/linoleic triglycerides, natural glycerides, as well as e.g. propylene glycol, dicaprylate/dicaprate and waxes, such as stearyl, stearate, oleyl oleate, isopropyl myristate
**Ceteareth 5-30, or other emulsifiers such as Polysorbate 20-80, sorbitane esters of fatty acids, fatty acid esters of PEG.
***Preservatives, e.g. paraben esters (methyl, ethyl, propyl, butyl), sorbic acid and/or benzoic acid.

### EXAMPLE 17

### Nasal Spray

A nasal spray suspension with the following composition is prepared and filled into a metered dose pocket sprayer:

TABLE 23
Nasal Spray in metered dose pocket sprayer

| Ingredients | mg/intranasal dose |
|---|---|
| Active compound | 0.3 to 2 mg per intranasal dose |
| Mixture with sorbitan trioleate, | |
| (d,l)-α-tocopherol | |

### EXAMPLE 18

### Aerosol

An aerosol suspension for inhalation with the following composition is prepared and filled into a metered dose inhaler:

TABLE 24
Inhaler Suspension in metered dose inhaler

| Ingredients | mg/inhaled dose |
|---|---|
| Active compound | 0.3 to 2 mg per inhaledl |
| | dose |
| Mixture with sorbitan trioleate and d,l-α- | |
| tocopherol and propellant tetrafluoroethane | |
| (HFA 134a) | |

### EXAMPLE 19

### Dry Powder for Inhaler

A dry powder inhaler is filled with the following mixture:

TABLE 25
Dry powder inhaler

| | | |
|---|---|---|
| Active compound (jet-milled, spray-dried) | 0.3-2.0 | mg per inhaled dose |
| Lactose monohydrate | 25 | mg |

### EXAMPLE 20

### Crystalline Suspension

A crystalline suspension is prepared for intra-articular injection, epidural injection or intrafocal infiltration as a slow release formulation. A suspension is also prepared for intravitreal injection for treating diabetic retinopathy

TABLE 26
Crystalline Suspension

| | |
|---|---|
| Active compound | 20-200 mg |
| The active compound is mixed with | |
| methylcellulose | |
| polysorbate | |
| antioxidants | |
| preservatives and | |
| distilled water ad 1 ml. | |

Examples for the Effect of an RXR Antagonist Administered Topically to the Skin of a Healthy Human Volunteer. Clinical Pilot Trial. Epicutaneous Application:

### EXAMPLE 21

### Effect of Topical Compound 15 on Healthy, Non-Inflamed Human Skin

Topical application of compounds to the skin is frequently handicapped by inflammation of human skin. Compound 15 (RXR antagonist) is therefore tested for its inflammation potential on human skin.

### Methods

In a clinical pilot trial on one healthy volunteer compound 15 is applied epicutaneously in a concentration of 1% for its inflammation potential compared to that of the strong inflammation inducing agent 9-cis RA in concentrations of 0.1%, 0.3% and 1%.

The compounds are solved or suspended in ethanol/propylene glycol (1:1). They are applied epicutaneously twice daily, 7 days a week, for two concecutive weeks. The site of application is an area of 3×3=9 cm² on the abdominal skin. The volume is 0.1 ml per application.

The following signs and symptoms of skin inflammation are recorded: Erythema, desquamation (scaling), pruritus, burning, pain, exsudation, edema, ulceration. They are classified on a scale of:
0=no signs or symptoms of skin inflammation
1=slight erythema, desquamation and pruritus
2=moderate erythema, desquamation and pruritus
3=marked erythema, desquamation, pruritus/burning
4=severe erythema, desquamation, pruritus, burning or even pain, edema, exsudation, ulceration

Treatment period lasts from day 1 to 14, the post-treatment observation period from day 15 to day 28.

### Results

9-cis RA is tested in three concentrations: 0.1%, 0.3% and 1.0%. During the first 9 days after start of treatment no signs or symptoms of skin inflammation are observed. Around the tenth and eleventh day, the symptoms become manifest in the form of slight erythema, desquamation and pruritus. These symptoms increase during days 12, 13 and 14, depending on the concentration, to moderate inflammation with 0.1 and 0.3% and to marked inflammation with 1.0%. Three days after discontinuation of treatment i.e. on day 17, the inflammation still persists and is concentration dependent: Marked inflammation (3) with 1%, moderate (2) with 0.3% and slight (1) with 0.1%. From day 18 on, inflammation decreases to 0, depending on the concentration, between days 18 and 22, i.e. 4 to 8 days after discontinuation of treatment.

Compound 15 is tested at a concentration of 1%. In contrast to 9-cis RA which induces a marked skin inflammation at 1% concentration, compound 15 is well tolerated with no skin inflammation at 1% concentration. Compound A does not induce any objective or subjective symptoms, neither during the treatment period, nor during the post-treatment observation period.

### Conclusion:

Compound 15 applied epicutaneously to human skin does not induce signs or symptoms of inflammation of the skin in a 28 days clinical pilot study.

### EXAMPLE 22

### Therapeutic Effect of Topical Compound 15 on Human Skin Inflammation Induced by Topical 9-cis Retinoic Acid (9-cis RA).

Compound 15 (RXR antagonist) applied epicutanously is tested on its anti-inflammatory effect on human skin, in which inflammation has been induced by topical 9-cis RA.

### Methods

In the following pilot clinical trial on one healthy volunteer (WB) the compounds are applied epicutaneously. 9-cis RA being known for its skin inflammation potential is used in concentrations of 0.1%, 0.3% and 1%. Compound 15 (RXR antagonist) concentration is 1%. The compounds are solved in ethanol/propylene glycol (1:1). 9-cis RA is applied twice daily, 7 days a week, for two consecutive weeks. The site of application is an area of 3×3=9 cm² on the abdominal skin. The volume is 0.1 ml per administration. The treatment with compound 15, administered twice daily in a concentration of 1%, is started on day 15 when treatment with 9-cis RA is discontinued. This treatment lasts from day 15 to day 22. For a correct evaluation comparative areas with inflammation induced by 9-cis RA are treated with the vehicle, ethanol/propylene glycol, from day 15 to 22. The post-treatment period lasts until day 28.

The signs and symptoms of skin inflammation are recorded on a 0-4 scale, as described in example 21.

For evaluation of the anti-inflammatory effect of compound 15, the sum of the daily inflammation scores from day 15 to day 28 is determined, as well as the time to complete disappearance of skin inflammation from day 15 on.

### Results (see Table 27)

### Anti-Inflammatory Effect of RXR Antagonist Compound 15 on 9-cis Retinoic Acid (9-cis RA)-Induced Skin Inflammation—Clinical Pilot Trial

| Induction of skin | Topical application of 9-cis RA 0.1%, 0.3% and 1% |
|---|---|
| inflammation: | |
| Therapy: | Topical application of compound 15: 1% |
| Comparison: | 9-cis RA 0.1%, 0.3% and 1% day 1-14, |
| | followed by vehicle, day 15-22 |
| | 9-cis RA 0.1%, 0.3% and 1% day 1-14, |
| | followed by compound 15: 1%, day 15-22 |
| Skin inflammation: | scale 0-4, daily determination from baseline to day |
| | 28 |

TABLE 27

| | Sum of daily | Time in days |
|---|---|---|
| | inflammation scores from | from day 15 to |
| | day 15 to disappearance | disappearance of |
| Retinoids | of inflammation | inflammation |
| 9-cis RA 0.1% | 6.5 | 8 |
| 9-cis RA 0.1% + | 2.5 | 3 |
| compound 15: 1% | | |
| 9-cis RA 0.3% | 14 | 13 |
| 9-cis RA 0.3% + | 5.5 | 6 |
| compound 15: 1% | | |
| 9-cis RA 1% | 14.5 | 9 |
| 9-cis RA 1% + | 11 | 8 |
| compound 15: 1% | | |

9-cis RA given topically exerts a significant skin inflammatory effect. The induction of inflammation is dependent on the concentration of 9-cis RA. A solution of 1% 9-cis RA provokes a much higher skin irritation than that of 0.1%.

After the treatment with 9-cis RA within the first 2 weeks, inflammation tends to decrease and to disappear between day 15 and day 23.

The time to disappearance of inflammation is markedly shortened when compound 15 in a concentration of 1% is applied between day 15 and day 22, compared with the vehicle control. In the case of 0.1% 9-cis RA induction of skin inflammation the time to disappearance of inflammation is 3 days, when compound 15 is administered in a 1% concentration, compared to 8 days in the case of vehicle application.

This anti-inflammatory effect is also evidenced by determination of the sum of daily inflammation scores from day 15 to day 28. By the treatment with 1% of compound 15, the sum of daily inflammation scores in the 0.1% 9-cis RA study is reduced to 2.5, compared to 6.5 by treatment with the vehicle control.

The study with 0.3% 9-cis RA gives rather similar effects as the study with 0.1% 9-cis RA. When inflammation is induced by the high concentration of 1% 9-cis RA the anti-inflammatory effect is less significant, with a reduction of the sum of daily inflammation scores from 14.5 to 11.

### EXAMPLE 23

### Anti-Inflammatory Effect of RXR Antagonist Compound 15 on 9-cis RA Induced Skin Inflammation—Comparison of Preventive and Therapeutic Effect of Compound 15

This human volunteer study represents an additional study with regard to example 22. Example 22 deals with a study wherein the therapeutic anti-inflammatory effect of compound 15 is demonstrated by the administration of topical compound 15 after the skin inflammation has been induced before, by topical administration of 9-cis RA. The present study is carried out for comparing the preventive and the therapeutic effect of the RXR antagonist compound 15 on skin inflammation induced by topical 9-cis RA.

### Methods

In this pilot clinical trial on one healthy volunteer (WB), the substance 9-cis RA and compound 15 are administered epicutaneously. The inflammation-inducing agent 9-cis RA is used in a concentration of 0.3%. The RXR antagonist compound 15 is applied in a concentration of 1%. The compounds are solved in ethanol/propylene glycol (1:1) and administered twice daily. The site of application is the abdominal skin, with various areas of 3×3=9 cm². Volume per application is 0.1 ml.

The following three clinical settings at different sites are chosen:
1. Induction of skin inflammation by 9-cis RA 9-cis RA is administered as a 0.3% solution twice daily from day 1 to day 14. The vehicle is administered from day 15 to disappearance of skin inflammation.
2. Prevention of 9-cis RA-induced skin inflammation by RXR antagonist Compound 15 9-cis RA solution of 0.3% is administered twice daily from day 1 to day 14, each time followed subsequently by application of compound 15 as a 1% solution.
3. Therapy of 9-cis RA-induced skin inflammation by RXR antagonist compound 15 9-cis RA solution of 0.3% is administered twice daily from day 1 to day 14. Compound 15 as a 1% solution is administered from day 15 until disappearance of skin inflammation.

The signs and symptoms of skin inflammation are recorded on a 0-4 scale, as described in example 21. The evaluation of the anti-inflammatory effect is based on the daily determination of the inflammation score (scale 0-4).

The following parameters serve as criteria for evaluation of the effect of 9-cis RA, the preventive effect of compound 15 and the therapeutic effect of compound 15.
1. Sum of the daily inflammation scores from day 1 to day 28. Total inflammation score.
2. Sum of the daily inflammation scores from day 15 to disappearance of skin inflammation.
3. Time in days from day 15 to disappearance of skin inflammation.

### Results (Table 28):

TABLE 28

| | | Sum of daily | |
|---|---|---|---|
| | Sum of daily | inflammation | |
| | inflammation | scores, | Time in days |
| | scores, day 1 | from day 15 to | from day 15 to |
| | to day 28. Total | disappearance | disappearance |
| | inflammation | of | of |
| Retinoids | score | inflammation | inflammation |
| 9-cis RA 0.3%, | 18 | 14 | 13 |
| day 1 to day 14 | | | |
| Prevention | 11 | 5 | 6 |
| 9-cis RA 0.3%, | | | |
| day 1 to day 14 | | | |
| compound 15: 1%, | | | |
| day 1 to day 14 | | | |
| Therapy | 9 | 5 | 6 |
| 9-cis RA 0.3%, | | | |
| day 1 to day 14 | | | |
| compound 15: 1%, | | | |
| day 15 to | | | |
| disappearance of | | | |
| inflammation | | | |

9-cis RA has a marked inflammatory effect on human skin. The total inflammation score is 18. The sum of daily inflammation scores from day 15 to complete disappearance is 14. 13 days are needed from day 15 on to complete disappearance of skin inflammation.

### Prevention of Skin Inflammation by the RXR Antagonist Compound 15

Compound 15 has a marked effect. All parameters for evaluation are influenced. In this prevention trial the total inflammation scores decrease from 18 to 11, the sum of daily inflammation scores from day 15 to disappearance of skin inflammation decreases from 14 to 5 and the time from day 15 to disappearance of skin inflammation from day 15 to disappearance of skin inflammation decreases from 13 days to 6 days.

### Therapy of Skin Inflammation by the RXR Antagonist Compound 15

Compound 15 has a marked anti-inflammatory effect in this therapeutic clinical trial. All parameters are reduced by 50% or more in comparison to the values of the inflammation-in-ducing agent 9-cis RA. Total inflammation score decreases from 18 to 9, the sum of daily inflammation scores from day 15 until disappearance of skin inflammation is reduced from 14 to 5 and the time from day 15 to disappearance of skin inflammation decreases from 13 to 6 days.

### Conclusion

The results of examples 22 and 23 represent a clinical proof of concept for the efficacy of the RXR antagonist compound 15 as an anti-inflammatory agent in prevention and therapy of inflammatory diseases, in particular inflammatory diseases of the skin.

### EXAMPLE 24

### Effect of Compound 15 Administered to the Skin of Healthy Volunteers where Skin Inflammation is Induced by Topical Application of Candidin (Extract of Candida Albicans) or UV-B Irradiation

Clinical phase I trial, approved by the Ethical Committee of the University Hospital of Geneva and by Swiss Drug Agency Swiss Medic.

In this clinical phase I trial, the anti-inflammatory effect of compound 15 and that of conventional therapy with topical corticosteroids or immunomodulatory macrolides are compared. In contrast to the examples 22 and 23, skin inflammation in example 24 is not induced by a retinoid agonist such as 9-cis retinoic acid. The anti-inflammatory effect of the retinoid antagonist compound 15 (RXR antagonist) can therefore not be considered merely as suppression of the toxic and inflammatory effect of a retinoid agonist.

Further evidence of the large difference between the inflammatory effect of a retinoid agonist and that of the other inflammation-inducing agents is the fact that their spectrum of toxic side effects is highly different. Retinoid agonists, when administered systemically, induce the typical hypervitaminosis A syndrome, manifesting itself in headache, flushes, cheilitis, conjunctivitis, various other mucocutaneous manifestations, musculoskeletal symptoms and laboratory abnormalities, such as elevation of transaminases, triglyerides and cholesterol. The skin inflammation-inducing agents used in example 24 do not induce this spectrum of toxic side effects. Thus example 24 is a clinical proof for the unexpected and non-obvious inventive general anti-inflammatory usefulness of the group of RXR antagonistic compounds.

### Methods:

Four healthy volunteers participate. Inclusion criteria are: Age above 18 years, male or female. They are informed, with written letter of consent. Exclusion criteria are: Preexistant dermatological diseases, known allergy to test agents.

Inflammation inducing agents: Candidin is administered intradermally (see also D. Poffet, Comparaison entre le pouvoir vaso-constricteur d'un corticoide topique et l'inhibition de la dermite à la candidine après intradermorèaction (IDR). Thèse, Universitè de Genève, 1984). UV-B rays are administered by a UV-B lamp.

Inflammation is measured quantitatively. The area of the inflamed skin is measured in cm². The thickness of the skin is monitored by Ultrasound at 20 MHz. Erythema is measured by colorimetric determination employing a Minolta CR 20.

The following anti-inflammatory agents are used:
Compound 15 (RXR antagonist): Lotion, concentration of 1% active ingredient dissolved in ethanol/PEG 400/water (3:1:1).
Corticosteroids: Diprosone® (Essex Pharma; active principle: betamethasone dipropionate) cream; Dermovate® (GlaxoSmithKline; active principle: clobetasol propionate) cream.
Macrolides: Protopic® (Fujisawa; active principle: tacrolimus) cream; Elidel® (Novartis; active principle: pimecrolimus) cream.

Plan of the study:

Area of administration: Six separate small skin areas on each forearm of every volunteer.
Day 1: Determination of skin thickness. Administration of the inflammatory agents (only on day 1). Administration of compound 15, corticosteroids or macrolides on the area where inflammatory agents had been placed immediately before. One area is treated by vehicle control or not treated at all.
Day 2: Determination of skin thickness, erythema and area of inflamed skin. Application of anti-inflammatory test substances.
Day 3: Determination of skin thickness, erythema and area of inflamed skin. Application of anti-inflammatory test substances.
Day 4: Determination of skin thickness, erythema and area of inflamed skin.

All determinations are recorded on a scale from 0 to 4:
0=no reduction
1=slight reduction (10 to 20%)
2=moderate reduction (21 to 40%)
3=marked reduction (41 to 70%)
4=very marked reduction (71 to 100%)
Compared to vehicle control.

### Results:

In the 4 volunteers a marked to very marked reduction of the inflammatory reaction is found when treated by corticosteroids or immunomodulatory macrolides. In the volunteers treated with topical compound 15, the anti-inflammatory effect is even superior to that of corticosteroids or macrolides. The reduction of the inflammatory reaction is very marked in all volunteers treated with topical compound 15. A further advantage of compound 15 is the fact that in earlier pilot trials compound 15 is devoid of any side effects (see also Example 21) on the skin, even when applied topically not only for 4 days but even for 14 to 20 days. This is not always the case with corticosteroids and immuno-modulatory macrolides.

Conclusion: Compound 15 administered topically has been demonstrated to be a clinically efficacious anti-inflammatory agent, superior to conventional therapy with topical corticosteroids or immunomodulatory macrolides. A further advantage of topical compound 15 is its lack of inducing adverse side effects on the skin. The results in the pilot trials of example 24 on humans correspond to the results achieved in the animal experiment reported in examples 6 and 7 given above.

The invention especially relates to the invention as hereinbefore described and as given in the claims which are enclosed in the description by reference herewith. In the description and claims, one or more up to all more general expressions can, independently of each other, be replaced by more specific corresponding expressions given in the description and the claims, respectively, thus defining more preferred embodiments of the invention.

1. A method of treating one or more diseases that are manifestations of a metabolic syndrome or one or more complications of the metabolic syndrome, the method including prevention, prophylaxix, therapy, or a combination thereof and comprising administering to a warm blooded animal or a human patient in need of such treatment one or more retinoid agonists, one or more retinoid antagonists, or one or more retinoid agonists and one or more retinoid antagonists, the retinoid agonists and retinoid antagonists comprising retinoids with selective Retinoid X Receptor (RXR) agonistic (RXR agonist) or antagonistic (RXR antagonist) activity, in a dose that is effective in said treatment,
the RXR agonists are selected from the group consisting of
Compound 1: (2E,4E)-3-methyl-5-[2-[(E)-2-(2,6,6-trimethyl-1-cyclohexen-1-yl)ethenyl]-1-cyclohepten-1-yl]-2,4-pentadienoic acid;
Compound 4: (2E,4E)-3-methyl-5-[(1RS,2RS)-2-(5,5,8,8-tetramethyl-5,6,7,8-tetrahrydro-naphthalen-2-yl)-cyclopropyl]-penta-2,4-dienoic acid;
Compound 5: (2E,4E)-3-Methyl-5-[2-[(E)-2-(2,6,6-trimethyl-1-cyclohexen-1-yl)ethenyl]-3-thienyl]-2,4-pentadienoic acid;
Compound 6: (2E,4E)-3-Methyl-5-[2-(5,5,8,8-tetramethyl-5,6,7,8-tetrahydro-naphthalen-2-yl)-cyclohex-1-enyl]-penta-2,4-dienoic acid;
Compound 7: (2E,4E)-3-methyl-5-[2-(5,5,8,8-tetramethyl-5,6,7,8-tetrahydro-naphthalen-2-yl)-phenyl]-penta-2,4-dienoic acid; and
Compound 10: (2E,4E)-3-Methyl-5-[(1S,2S)-2-(5,5,8,8-tetramethyl 5,6,7,8-tetrahydro-naphthalen-2-yl)-cyclopropyl]-penta-2,4-dienoic acid;
and the RXR antagonists are selected from the group consisting of
Compound 15: (2E,4E,6Z)-7-[2-butoxy-3,5-bis(1,1-dimethylethyl)phenyl]-3-methyl-2,4,6-octatrienoic acid;
Compound 16: (2E,4E)-(1RS,2RS)-5-[2-(3,5-Di-tert-butyl-2-butoxyphenyl)-cyclopropyl]-3-methyl-penta-2, 4-dienoic acid;
Compound 17: (2E,4E)-(1RS,2RS)-5-[2-(3,5-Di-tert-butyl-2-ethoxy-phenyl)-cyclopropyl]-3-methyl-penta-2,4-dienoic acid;
Compound 18: (2E,4E)-3-Methyl-5-[2,6,6-trimethyl-cyclohex-1-enylethynyl)-cyclohept-1-enyl]-penta-2,4-dienoic acid; and
Compound 21: (2E,4E,6Z)-7-[3,5-Bis(1,1-dimethylethyl)-2-ethoxyphenyl]-3-methyl-2,4,6-octatrienoic acid.

2. The method according to claim 1, where the step of administering includes oral administration, topical administration, or oral and topical administration.

3. The method according to claim 1, where the one or more diseases are selected from the group consisting of diabetes type II, obesity, dyslipidemia, hypertension and polyneuropathy.

4. The method according to claim 1 where the one or more complications are selected from the group consisting of cardiovascular diseases, atherosclerosis.

5. The method according to claim 1, including administration of one of the RXR agonists, one of the RXR antagonists, or one of the RXR agonists and one of the RXR antagonists with one peroxisome proliferator activated receptor (PPAR) ligand.

6. The method according to claim 1 wherein the PPAR ligand is selected from the group consisting of fibrates, fibrates from clofibrate, fibrates from fenofibrate, glitazones, glitazones from rosiglitazone, and glitazones from pioglitazone.

7. The method according to claim 1 wherein the RXR agonist or the RXR antagonist is present in at least one form selected from the group consisting of a free form, a pharmaceutically acceptable salt, an amide, an ester and a pharmaceutically acceptable salt of an ester or amide.

8. A kit comprising a fixed combination including a PPAR ligand in combination with an RXR antagonist, an RXR agonist, or an RXR antagonist and an RXR agonist, the RXR agonist selected from the group consisting of
Compound 1: (2E,4E)-3-methyl-5-[2-[(E)-2-(2,6,6-trimethyl-1-cyclohexen-1-yl)ethenyl]-1-cyclohepten-1-yl]-2,4-pentadienoic acid;
Compound 4: (2E,4E)-3-methyl-5-[(1RS,2RS)-2-(5,6,8,8-tetramethyl-5,6,7,8-tetrahrydro-naphthalen-2-yl)-cyclopropyl]-penta-2,4-dienoic acid:
Compound 5: (2E,4E)-3-Methyl-5-[2-[(E)-2-(2,6,6-trimethyl-1-cyclohexen-1-yl)ethenyl]-3-thienyl]-2,4-pentadienoic acid;
Compound 6: (2E,4E)-3-Methyl-5-[2-(5,5,8,8-tetramethyl-5,6,7,8-tetrahydro-naphthalen-2-yl)-cyclohex-1-enyl]-penta-2,4-dienoic acid;
Compound 7: (2E,4E)-3-methyl-5-[2-(5,5,8,8-tetramethyl-5,6,7,8-tetrahydro-naphthalen-2-yl)-phenyl]-penta-2,4-dienoic acid; and
Compound 10: (2E,4E)-3-Methyl-5-[(1S,2S)-2-(5,5,8,8-tetramethyl]5,6,7,8-tetrahydro-naphthalen-2-yl)-cyclopropyl]-penta-2,4-dienoic acid;
and the RXR antagonist selected from the group consisting of
Compound 15: (2E,4E,6Z)-7-[2-butoxy-3,5-bis(1,1-dimethylethyl)phenyl]-3-methyl-2,4,6-octatrienoic acid:
Compound 16: (2E,4E)-(1RS,2RS)-5-[2-(3,5-Di-tert-butyl-2-butoxyphenyl)-cyclopropyl]-3-methyl-penta-2,4-dienoic acid;
Compound 17: (2E,4E)-(1RS,2RS)-5-[2-(3,5-Di-tert-butyl-2-ethoxy- phenyl)-cyclopropyl]-3-methyl-penta-2,4-dienoic acid;
Compound 18: (2E,4E)-3-Methyl-5-[2,6,6-trimethyl-cyclohex-1-enylethynyl)-cyclohept-1-enyl]-penta-2,4-dienoic acid; and
Compound 21: (2E,4E,6Z)-7-[3,5-Bis(1,1-dimethylethyl)-2- ethoxyphenyl]-3-methyl-2,4,6-octatrienoic acid.

9. The kit according to claim 8, wherein the relative weight amounts of the RXR agonists and/or the RXR antagonists relatively to the PPAR ligand is in a range selected from the group consisting of 1:50, 1:5, 50:1, and 5:1.

10. A composition comprising one or more RXR agonists, one or more RXR antagonists or one or more RXR agonists and one or more RXR antagonists the one or more RXR agonists selected from the group consisting of
Compound 1: (2E,4E)-3-methyl-5-[2-[(E)-2-(2,6,6-trimethyl-1-cyclohexen-1-yl)ethenyl]-1-cyclohepten-1-yl]-2,4-pentadienoic acid:
Compound 4: (2E,4E)-3-methyl-5-[(1RS,2RS)-2-(5,6,8,8-tetramethyl-5,6,7,8-tetrahrydro-naphthalen-2-yl)-cyclopropyl]-penta-2,4-dienoic acid:
Compound 5: (2E,4E)-3-Methyl-5-[2-[(E)-2-(2,6,6-trimethyl-1-cyclohexen-1-yl)ethenyl]-3-thienyl]-2,4-pentadienoic acid;
Compound 6: (2E,4E)-3-Methyl-5-[2-(5,5,8,8-tetramethyl-5,6,7,8-tetrahydro-naphthalen-2-yl)-cyclohex-1-enyl]-penta-2,4-dienoic acid;
Compound 7: (2E,4E)-3-methyl-5-[2-(5,5,8,8-tetramethyl-5,6,7,8-tetrahydro-naphthalen-2-yl)-phenyl]-penta-2,4g-dienoic acid; and
Compound 10: (2E,4E)-3-Methyl-5-[(1S,2S)-2-(5,5,8,8-tetramethyl 5,6,7,8-tetrahydro-naphthalen-2-yl)-cyclopropyl]-penta-2,4-dienoic acid;
and the one or more RXR antagonists selectected from the group consisting of
Compound 15: (2E,4E,6Z)-7-[2-butoxy-3,5-bis(1,1-dimethylethyl)phenyl]-3-methyl-2,4,6-octatrienoic acid;
Compound 16: (2E,4E)-(1RS,2RS)-5-[2-(3,5-Di-tert-butyl-2- butoxyphenyl)-cyclopropyl]-3-methyl-penta-2,4-dienoic acid;
Compound 17: (2E,4E)-(1RS,2RS)-5-[2-(3,5-Di-tert-butyl-2-ethoxy-phenyl)-cyclopropyl]-3-methyl-penta-2,4-dienoic acid;
Compound 18: (2E,4E)-3-Methyl-5-[2,6,6-trimethyl-cyclohex-1-enylethynyl)-cyclohept-1-enyl]-penta-2,4-dienoic acid; and
Compound 21: (2E,4E,6Z)-7-[3,5-Bis(1,1-dimethylethyl)-2-ethoxyphenyl]-3-methyl-2,4,6-octatrienoic acid;
where each of the RXR agonists and RXR antagonists independently of the others, is present in free form or in a pharmaceutically acceptable amide, ester or a pharmaceutically acceptable salt of the ester or the amide, said composition also comprising at least one pharmaceutically acceptable carrier.

11. A composition according to claim 10 further comprising one or more PPAR ligands.

12. The composition according to claim 11 in the form of a fixed combination.

13. A kit comprising in separate containers one or more PPAR ligands and one or more RXR agonists and/or one or more RXR antagonists the one or more RXR agonists selected from the group consisting of
Compound 1: (2E,4E)-3-methyl-5-[2-[(E)-2-(2,6,6-trimethyl-1-cyclohexen-1-yl)ethenyl]-1-cyclohepten-1-yl]-2,4-pentadienoic acid;
Compound 4: (2E,4E)-3-methyl-5-[(1RS,2RS)-2-(5,6,8,8-tetramethyl-5,6,7,8-tetrahrydro-naphthalen-2-yl)-cyclopropyl]-penta-2,4-dienoic acid;
Compound 5: (2E,4E)-3-Methyl-5-[2-[(E)-2-(2,6,6-trimethyl-1-cyclohexen-1-yl)ethenyl]-3-thienyl]-2,4-pentadienoic acid;
Compound 6: (2E,4E)-3-Methyl-5-[2-(5,5,8,8-tetramethyl-5,6,7,8-tetrahydro-naphthalen-2-yl)-cyclohex-1-enyl]-penta-2,4-dienoic acid;
Compound 7: (2E,4E)-3-methyl-5-[2-(5,5,8,8-tetramethyl-5,6,7,8-tetrahydro-naphthalen-2-yl)-phenyl]-penta-2,4-dienoic acid; and
Compound 10: (2E,4E)-3-Methyl-5-[(1S,2S)-2-(5,5,8,8-tetramethyl 5,6,7,8-tetrahydro-naphthalen-2-yl)-cyclopropyl]-penta-2,4-dienoic acid;
and the one or more RXR antagonists selectected from the group consisting of
Compound 15: (2E,4E,6Z)-7-[2-butoxy-3,5-bis(1,1-dimethylethyl)phenyl]-3-methyl-2,4,6-octatrienoic acid;
Compound 16: (2E,4E)-(1RS,2RS)-5-[2-(3,5-Di-tert-butyl-2- butoxyphenyl)-cyclopropyl]-3-methyl-penta-2,4-dienoic acid;
Compound 17: (2E,4E)-(1RS,2RS)-5-[2-(3,5-Di-tert-butyl-2-ethoxy-phenyl)-cyclopropyl]-3-methyl-penta-2,4-dienoic acid;
Compound 18: (2E,4E)-3-Methyl-5-[2,6,6-trimethyl-cyclohex-1-enylethynyl)-cyclohept-1-enyl]-penta-2,4-dienoic acid; and
Compound 21: (2E,4E,6Z)-7-[3,5-Bis(1,1-dimethylethyl)-2-ethoxyphenyl]-3-methyl-2,4,6-octatrienoic acid; where each of the RXR agonists and RXR antagonists, independently of the others, is present in free form or in a pharmaceutically acceptable amide, ester or a pharmaceutically acceptable salt of the ester or the amide, said composition also comprising at least one pharmaceutically acceptable carrier;
The kit including instructions for administration of the one or more PPAR ligands and the one or more RXR agonists and/or RXR antagonists in a manner selected from the group consisting of at separate time periods, overlapping time periods, at the same time, in separate formulations, and in combination with each other.

14. (canceled)

15. (canceled)

16. The method of claim 1 further comprising administering one or more peroxisome proliferator activated receptor (PPAR) ligands.

17. The method according to claim 16 wherein the PPAR ligand is present in at least one form selected from the group consisting of a free form, a pharmaceutically acceptable salt, an amide, an ester and a pharmaceutically acceptable salt of an ester or amide.

18. The method according to claim 16, wherein the relative weight amounts of the RXR agonists and/or RXR antagonists relative to the one or more PPAR ligands is in a range selected from the group consisting of 1:50, 1:5, 50:1, and 5:1.

19. The method of claim 3, wherein the one or more diseases are linked with a high risk of cardiovascular diseases and wherein the method further includes treatment of cardiovascular disease.

20. A kit comprising separate containers of a PPAR ligand and one or more RXR antagonist, one or more RXR agonist, or one or more RXR antagonist and one or more RXR agonist;
the kit further comprising an indication that the contents of the separate containers can be combined and administered to a patient to treat one or more diseases that are manifestations of a metabolic syndrome or one or more complications of the metabolic syndrome; the one or more RXR agonists are selected from the group consisting of
Compound 1: (2E,4E)-3-methyl-5-[2-[(E)-2-(2,6,6-trimethyl-1-cyclohexen-1-yl)ethenyl]-1-cyclohepten-1-yl]-2,4-pentadienoic acid;
Compound 4: (2E,4E)-3-methyl-5-[(1RS,2RS)-2-(5,5,8,8-tetramethyl-5,6,7,8-tetrahrydro-naphthalen-2-yl)-cyclopropyl]-penta-2,4-dienoic acid;
Compound 5: (2E,4E)-3-Methyl-5-[2-[(E)-2-(2,6,6-trimethyl-1-cyclohexen-1-yl)ethenyl]-3-thienyl]-2,4-pentadienoic acid;
Compound 6: (2E,4E)-3-Methyl-5-[2-(5,5,8,8-tetramethyl-5,6,7,8-tetrahydro-naphthalen-2-yl)-cyclohex-1-enyl]-penta-2,4-dienoic acid;
Compound 7: (2E,4E)-3-methyl-5-[2-(5,5,8,8-tetramethyl-5,6,7,8-tetrahydro-naphthalen-2-yl)-phenyl]-penta-2,4-dienoic acid; and
Compound 10: (2E,4E)-3-Methyl-5-[(1S,2S)-2-(5,5,8,8-tetramethyl 5,6,7,8-tetrahydro-naphthalen-2-yl)-cyclopropyl]-penta-2,4-dienoic acid;
and the one or more RXR antagonists are selected from the group consisting of
Compound 15: (2E,4E,6Z)-7-[2-butoxy-3,5-bis(1,1-dimethylethyl)phenyl]-3-methyl-2,4,6-octatrienoic acid;
Compound 16: (2E,4E)-(1RS,2RS)-5-[2-(3,5-Di-tert-butyl-2-butoxyphenyl)-cyclopropyl]-3-methyl-penta-2,4-dienoic acid;
Compound 17: (2E,4E)-(1RS,2RS)-5-[2-(3,5-Di-tert-butyl-2-ethoxy-phenyl)-cyclopropyl]-3-methyl-penta-2,4-dienoic acid;
Compound 18: (2E,4E)-3-Methyl-6-[2,6,6-trimethyl-cyclohex-1-enylethynyl)-cyclohept-1-enyl]-penta-2,4-dienoic acid; and
Compound 21: (2E,4E,6Z)-7-[3,5-Bis(1,1-dimethylethyl)-2-ethoxyphenyl]-3-methyl-2,4,6-octatrienoic acid.
